(19) United States
(12) Patent Application Publication
(10) Pub. No.: US 2018/0344741 A1
(43) Pub. Date: Dec. 6, 2018

(54) ANTIDIABETIC MEDICATIONS

(71) Applicant: Boehringer Ingelheim International Gmbh, Ingelheim am Rhein (DE)

(72) Inventors: Eva GRAEFE-MODY, Ingelheim am Rhein (DE); Hans-Juergen WOERLE, Grandvaus VD (CH)

(21) Appl. No.: 16/059,413

(22) Filed: Aug. 9, 2018

Related U.S. Application Data
(63) Continuation of application No. 15/444,362, filed on Feb. 28, 2017.
Application No. 15/444,362 is a continuation of application No. 14/578,552, filed on Dec. 22, 2014, now abandoned.
Application No. 14/578,552 is a continuation of application No. 13/148,065, filed on Sep. 27, 2011, now abandoned.

(30) Foreign Application Priority Data
Feb. 13, 2009 (EP) 09152869.5

(51) Int. Cl. A61K 31/522; A61K 47/26; A61K 9/00; A61K 9/20; A61K 45/06; A61K 31/155; A61K 9/48

(52) U.S. Cl. CPC A61K 31/522; A61K 47/26; A61K 9/0019; A61K 9/2013; A61K 9/2018; A61K 9/2027; A61K 2300/00; A61K 45/06; A61K 31/155; A61K 9/4866; A61K 9/4858; A61K 9/2059

(57) ABSTRACT

Methods of using antidiabetic medications which are suitable in the treatment or prevention of one or more conditions selected from type 1 diabetes mellitus, type 2 diabetes mellitus, impaired glucose tolerance, and hyperglycemia, among others.

### TECHNICAL FIELD OF THE INVENTION

The invention relates to DPP-4 inhibitors which are suitable in the treatment or prevention of one or more conditions selected from type 1 diabetes mellitus, type 2 diabetes mellitus, impaired glucose tolerance, impaired fasting blood glucose and hyperglycemia inter alia, as well as to a pharmaceutical composition or combination comprising such a DPP-4 inhibitor as defined herein and optionally one or more other active substances, its use in the therapy of metabolic disorders and, particularly, as antidiabetic medication.

Furthermore the invention relates to methods
for preventing, slowing progression of, delaying, or treating a metabolic disorder;
for improving glycemic control and/or for reducing of fasting plasma glucose, of postprandial plasma glucose and/or of glycosylated hemoglobin HbA1c;
for preventing, slowing, delaying or reversing progression from impaired glucose tolerance, impaired fasting blood glucose, insulin resistance and/or from metabolic syndrome to type 2 diabetes mellitus;
for preventing, slowing progression of, delaying or treating of a condition or disorder selected from the group consisting of complications of diabetes mellitus;
for reducing body weight and/or body fat or preventing an increase in body weight and/or body fat or facilitating a reduction in body weight and/or body fat;
for preventing or treating the degeneration of pancreatic beta cells and/or for improving and/or restoring or protecting the functionality of pancreatic beta cells and/or restoring the functionality of pancreatic insulin secretion;
for preventing, slowing, delaying or treating diseases or conditions attributed to an abnormal accumulation of liver or ectopic fat;
for maintaining and/or improving the insulin sensitivity and/or for treating or preventing hyperinsulinemia and/or insulin resistance;
for preventing, slowing progression of, delaying, or treating new onset diabetes after transplantation (NODAT) and/or post-transplant metabolic syndrome (PTMS);
for preventing, delaying, or reducing NODAT and/or PTMS associated complications including micro- and macrovascular diseases and events, graft rejection, infection, and death;
for treating hyperuricemia and hyperuricemia associated conditions;

in patients in need thereof characterized in that a DPP-4 inhibitor as defined hereinafter is administered, optionally in combination with one or more other active substances.

In addition, the present invention relates to the use of a DPP-4 inhibitor for the manufacture of a medicament for use in a method as described hereinbefore and hereinafter.

The invention also relates to a use of a pharmaceutical composition or combination according to this invention for the manufacture of a medicament for use in a method as described hereinbefore and hereinafter.

The invention also relates to the DPP-4 inhibitors as defined herein for use in a method as described hereinbefore and hereinafter, said method comprising administering the DPP-4 inhibitor, optionally in combination with one or more other active substances (e.g. which may selected from those mentioned herein), to the patient.

### BACKGROUND OF THE INVENTION

Type 2 diabetes is an increasingly prevalent disease that due to a high frequency of complications leads to a significant reduction of life expectancy. Because of diabetes-associated microvascular complications, type 2 diabetes is currently the most frequent cause of adult-onset loss of vision, renal failure, and amputations in the industrialized world. In addition, the presence of type 2 diabetes is associated with a two to five fold increase in cardiovascular disease risk.

After long duration of disease, most patients with type 2 diabetes will eventually fail on oral therapy and become insulin dependent with the necessity for daily injections and multiple daily glucose measurements.

The UKPDS (United Kingdom Prospective Diabetes Study) demonstrated that intensive treatment with metformin, sulfonylureas or insulin resulted in only a limited improvement of glycemic control (difference in HbA1c ˜0.9%). In addition, even in patients within the intensive treatment arm glycemic control deteriorated significantly over time and this was attributed to deterioration of β-cell function. Importantly, intensive treatment was not associated with a significant reduction in macrovascular complications, i.e. cardiovascular events. Therefore many patients with type 2 diabetes remain inadequately treated, partly because of limitations in long term efficacy, tolerability and dosing inconvenience of existing antihyperglycemic therapies.

Oral antidiabetic drugs conventionally used in therapy (such as e.g. first- or second-line, and/or mono- or (initial or add-on) combination therapy) include, without being restricted thereto, metformin, sulphonylureas, thiazolidinediones, glinides and α-glucosidase inhibitors.

Non-oral antidiabetic drugs conventionally used in therapy (such as e.g. first- or second-line, and/or mono- or (initial or add-on) combination therapy) include, without being restricted thereto, GLP-1 or GLP-1 analogues, and insulin or insulin analogues.

The high incidence of therapeutic failure is a major contributor to the high rate of long-term hyperglycemia-associated complications or chronic damages (including micro- and makrovascular complications such as e.g. diabetic nephrophathy, retinopathy or neuropathy, or cardiovascular complications) in patients with type 2 diabetes.

Therefore, there is an unmet medical need for methods, medicaments and pharmaceutical compositions or combinations with a good efficacy with regard to glycemic control, with regard to disease-modifying properties and with regard to reduction of cardiovascular morbidity and mortality while at the same time showing an improved safety profile.

DPP-4 inhibitors represent another novel class of agents that are being developed for the treatment or improvement in glycemic control in patients with type 2 diabetes.

For example, DPP-4 inhibitors and their uses are disclosed in WO 2002/068420, WO 2004/018467, WO 2004/018468, WO 2004/018469, WO 2004/041820, WO 2004/046148, WO 2005/051950, WO 2005/082906, WO 2005/063750, WO 2005/085246, WO 2006/027204, WO 2006/029769, WO2007/014886; WO 2004/050658, WO 2004/111051, WO 2005/058901, WO 2005/097798; WO 2006/068163, WO 2007/071738, WO 2008/017670; WO 2007/128724, WO 2007/128721 or WO 2007/128761, or WO 2009/121945.

### AIM OF THE PRESENT INVENTION

The aim of the present invention is to provide a medication and/or method for preventing, slowing progression of, delaying or treating a metabolic disorder, in particular of type 2 diabetes mellitus. A further aim of the present invention is to provide a medication and/or method for improving glycemic control in a patient in need thereof, in particular in patients with type 2 diabetes mellitus.

Another aim of the present invention is to provide a medication and/or method for improving glycemic control in a patient with insufficient glycemic control despite monotherapy with an antidiabetic drug, for example metformin, or despite combination therapy with two or three antidiabetic drugs.

Another aim of the present invention is to provide a medication and/or method for preventing, slowing or delaying progression from impaired glucose tolerance (IGT), impaired fasting blood glucose (IFG), insulin resistance and/or metabolic syndrome to type 2 diabetes mellitus.

Yet another aim of the present invention is to provide a medication and/or method for preventing, slowing progression of, delaying or treating of a condition or disorder from the group consisting of complications of diabetes mellitus.

A further aim of the present invention is to provide a medication and/or method for reducing the weight or preventing an increase of the weight in a patient in need thereof.

Another aim of the present invention is to provide a medication with a high efficacy for the treatment of metabolic disorders, in particular of diabetes mellitus, impaired glucose tolerance (IGT), impaired fasting blood glucose (IFG), and/or hyperglycemia, which has good to very good pharmacological and/or pharmacokinetic and/or physicochemical properties.

Further aims of the present invention become apparent to the one skilled in the art by description hereinbefore and in the following and by the examples.

### SUMMARY OF THE INVENTION

Within the scope of the present invention it has now surprisingly been found that DPP-4 inhibitors as defined herein as well as pharmaceutical compositions or combinations comprising a DPP-4 inhibitor as defined herein and optionally one or more other active substances can advantageously be used for preventing, slowing progression of, delaying (e.g. delaying the onset) or treating a metabolic disorder, in particular for improving glycemic control in patients. This opens up new therapeutic possibilities in the treatment and prevention of type 2 diabetes mellitus, overweight, obesity, complications of diabetes mellitus and of neighboring disease states.

Therefore, in a first aspect the present invention provides a pharmaceutical composition or combination comprising

(a) a DPP-4 inhibitor, and, optionally,

(b) a second antidiabetic agent selected from the group G3 consisting of biguanides (particularly metformin), thiazolidindiones, sulfonylureas, glinides, inhibitors of alpha-glucosidase and GLP-1 analogues, and, optionally,

(c) a third antidiabetic agent being different from (b) selected from the group G3 consisting of biguanides (particularly metformin), thiazolidindiones, sulfonylureas, glinides, inhibitors of alpha-glucosidase and GLP-1 analogues, or a pharmaceutically acceptable salt thereof.

In a subaspect the present invention provides a pharmaceutical composition or combination comprising

(a) a DPP-4 inhibitor, and, optionally,

(b) a second antidiabetic agent selected from the group G3 consisting of biguanides (particularly metformin), thiazolidindiones, sulfonylureas, glinides, inhibitors of alpha-glucosidase and GLP-1 analogues, and, optionally,

(c) a third antidiabetic agent being different from (b) selected from the group consisting of metformin, a sulfonylurea, pioglitazone, rosiglitazone, repaglinide, nateglinide, acarbose, voglibose, miglitol and a GLP-1 analogue, or a pharmaceutically acceptable salt thereof.

In another subaspect the present invention provides a pharmaceutical composition or combination comprising

(a) a DPP-4 inhibitor, and, optionally,

(b) a second antidiabetic agent selected from the group consisting of metformin, a sulfonylurea, pioglitazone, rosiglitazone, repaglinide, nateglinide, acarbose, voglibose, miglitol and a GLP-1 analogue, and, optionally,

(c) a third antidiabetic agent being different from (b) selected from the group G3 consisting of biguanides (particularly metformin), thiazolidindiones, sulfonylureas, glinides, inhibitors of alpha-glucosidase and GLP-1 analogues,

or a pharmaceutically acceptable salt thereof.

In a further subaspect the present invention provides a pharmaceutical composition or combination comprising

(a) a DPP-4 inhibitor, and, optionally,

(b) a second antidiabetic agent selected from the group consisting of metformin, a sulfonylurea and pioglitazone, and, optionally,

(c) a third antidiabetic agent being different from (b) selected from the group consisting of metformin, a sulfonylurea, pioglitazone, rosiglitazone, repaglinide, nateglinide, acarbose, voglibose, miglitol and a GLP-1 analogue,

or a pharmaceutically acceptable salt thereof.

In a further subaspect the present invention provides a pharmaceutical composition or combination comprising

(a) a DPP-4 inhibitor, and, optionally,

(b) a second antidiabetic agent selected from the group consisting of metformin, a sulfonylurea, pioglitazone, rosiglitazone, repaglinide, nateglinide, acarbose, voglibose, miglitol and a GLP-1 analogue, and, optionally,

(c) a third antidiabetic agent being different from (b) selected from the group consisting of metformin, a sulfonylurea and pioglitazone,

or a pharmaceutically acceptable salt thereof.

In a yet further subaspect the present invention provides a pharmaceutical composition or combination comprising

(a) a DPP-4 inhibitor, and, optionally,

(b) a second antidiabetic agent selected from the group consisting of metformin and pioglitazone, and, optionally,

(c) a third antidiabetic agent being different from (b) selected from the group consisting of metformin, a sulfonylurea and pioglitazone,

or a pharmaceutically acceptable salt thereof.

In a yet further subaspect the present invention provides a pharmaceutical composition or combination comprising

(a) a DPP-4 inhibitor, and, optionally,

(b) a second antidiabetic agent selected from the group consisting of metformin, a sulfonylurea and pioglitazone, and, optionally,

(c) a third antidiabetic agent being different from (b) selected from the group consisting of metformin and pioglitazone,

or a pharmaceutically acceptable salt thereof.

When—besides the second anidiabetic agent—a third antidiabetic agent is chosen, said third antidiabetic agent is preferably chosen from another class than the second antidiabetic agent. Thus, it is to be understood that the second and the third antidiabetic agent are different, and preferably they are from different classes (e.g. when the second antidiabetic agent is chosen from the biguanide class, the third antidiabetic agent is preferably chosen from another class). Classes of antidiabetic agents are mentioned above, e.g. biguanide class, thiazolidindione class, sulfonylurea class, glinide class, alpha-glucosidase inhibitor class, GLP-1 analogue class, etc.

An embodiment of this invention refers to monotherapy with a DPP-4 inhibitor as defined herein and/or to pharmaceutical compositions comprising a DPP-4 inhibitor as sole active ingredient.

Within combinations and/or combination therapy according to this invention, a particular embodiment refers to dual combinations and/or dual therapy; another embodiment refers to triple combinations and/or triple therapy.

According to another aspect of the invention, there is provided a method for preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity and metabolic syndrome in a patient in need thereof characterized in that a DPP-4 inhibitor and, optionally, a second and, optionally, a third antidiabetic agent as defined hereinbefore and hereinafter are administered, for example in combination, to the patient.

According to another aspect of the invention, there is provided a method for improving glycemic control and/or for reducing of fasting plasma glucose, of postprandial plasma glucose and/or of glycosylated hemoglobin HbA1c in a patient in need thereof characterized in that a DPP-4 inhibitor and, optionally, a second and, optionally, a third antidiabetic agent as defined hereinbefore and hereinafter are administered, for example in combination, to the patient.

The pharmaceutical composition according to this invention may also have valuable disease-modifying properties with respect to diseases or conditions related to impaired glucose tolerance (IGT), impaired fasting blood glucose (IFG), insulin resistance and/or metabolic syndrome.

According to another aspect of the invention, there is provided a method for preventing, slowing, delaying or reversing progression from impaired glucose tolerance (IGT), impaired fasting blood glucose (IFG), insulin resistance and/or from metabolic syndrome to type 2 diabetes mellitus in a patient in need thereof characterized in that a DPP-4 inhibitor and, optionally, a second and, optionally, a third antidiabetic agent as defined hereinbefore and hereinafter are administered, for example in combination, to the patient.

As by the use of a pharmaceutical composition or combination according to this invention, an improvement of the glycemic control in patients in need thereof is obtainable, also those conditions and/or diseases related to or caused by an increased blood glucose level may be treated.

According to another aspect of the invention, there is provided a method for preventing, slowing the progression of, delaying or treating of a condition or disorder selected from the group consisting of complications of diabetes mellitus such as cataracts and micro- and macrovascular diseases, such as nephropathy, retinopathy, neuropathy, learning and memory impairment, neurodegenerative or cognitive disorders, cardio- or cerebrovascular diseases, tissue ischaemia, diabetic foot or ulcus, arteriosclerosis, hypertension, endothelial dysfunction, myocardial infarction, accute coronary syndrome, unstable angina pectoris, stable angina pectoris, stroke, peripheral arterial occlusive disease, cardiomyopathy, heart failure, heart rhythm disorders and vascular restenosis, in a patient in need thereof characterized in that a DPP-4 inhibitor and, optionally, a second and, optionally, a third antidiabetic agent as defined hereinbefore and hereinafter are administered, for example in combination, to the patient. In particular one or more aspects of diabetic nephropathy such as hyperperfusion, proteinuria and albuminuria (e.g. micro- or macroalbuminuria) may be treated, their progression slowed or their onset delayed or prevented. The term “tissue ischaemia” particularly comprises diabetic macroangiopathy, diabetic microangiopathy, impaired wound healing and diabetic ulcer. The terms “micro- and macrovascular diseases” and “micro- and macrovascular complications” are used interchangeably in this application.

In an embodiment of the present invention, by the administration of a pharmaceutical composition or combination according to this invention no gain in weight or even a reduction in body weight is the result.

According to another aspect of the invention, there is provided a method for reducing body weight and/or body fat or preventing an increase in body weight and/or body fat or facilitating a reduction in body weight and/or body fat in a patient in need thereof characterized in that a DPP-4 inhibitor and, optionally, a second and, optionally, a third antidiabetic agent as defined hereinbefore and hereinafter are administered, for example in combination, to the patient.

In an embodiment of the present invention, by an administration of a pharmaceutical composition or combination according to this invention a beta-cell degeneration and a decline of beta-cell functionality such as for example apoptosis or necrosis of pancreatic beta cells can be delayed or prevented. Furthermore, the functionality of pancreatic cells can be improved or restored, and the number and size of pancreatic beta cells increased. It may be shown that the differentiation status and hyperplasia of pancreatic beta-cells disturbed by hyperglycemia can be normalized by treatment with a pharmaceutical composition according to this invention.

According to another aspect of the invention, there is provided a method for preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion in a patient in need thereof characterized in that a DPP-4 inhibitor and, optionally, a second and, optionally, a third antidiabetic agent as defined hereinbefore and hereinafter are administered, for example in combination, to the patient.

In an embodiment of the present invention, by the administration of a pharmaceutical composition or combination according to the present invention, an abnormal accumulation of ectopic fat, in particular in the liver, may be reduced or inhibited.

According to another aspect of the present invention, there is provided a method for preventing, slowing, delaying or treating diseases or conditions attributed to an abnormal accumulation of liver or ectopic fat in a patient in need thereof characterized in that a DPP-4 inhibitor and, optionally, a second and, optionally, a third antidiabetic agent as defined hereinbefore and hereinafter are administered, for example in combination, to the patient.

Diseases or conditions which are attributed to an abnormal accumulation of liver or ectopic fat are particularly selected from the group consisting of general fatty liver, non-alcoholic fatty liver (NAFL), non-alcoholic steatohepatitis (NASH), hyperalimentation-induced fatty liver, diabetic fatty liver, alcoholic-induced fatty liver or toxic fatty liver, particularly non-alcoholic fatty liver disease (NAFLD), including hepatic steatosis, non-alcoholic steatohepatitis (NASH) and/or liver fibrosis.

According to a further aspect of the present invention, there is provided a method for preventing, slowing the progression, delaying, attenuating, treating or reversing hepatic steatosis, (hepatic) inflammation and/or an abnormal accumulation of liver fat in a patient in need thereof characterized in that a DPP-4 inhibitor and, optionally, a second and, optionally, a third antidiabetic agent as defined hereinbefore and hereinafter are administered, for example in combination, to the patient.

Another aspect of the invention provides a method for maintaining and/or improving the insulin sensitivity and/or for treating or preventing hyperinsulinemia and/or insulin resistance in a patient in need thereof characterized in that a DPP-4 inhibitor and, optionally, a second and, optionally, a third antidiabetic agent as defined hereinbefore and hereinafter are administered, for example in combination, to the patient.

According to another aspect of the invention, there is provided a method for preventing, slowing progression of, delaying, or treating new onset diabetes after transplantation (NODAT) and/or post-transplant metabolic syndrome (PTMS) in a patient in need thereof characterized in that a DPP-4 inhibitor and, optionally, a second and, optionally, a third antidiabetic agent as defined hereinbefore and hereinafter are administered, for example in combination, to the patient.

According to a further aspect of the invention, there is provided a method for preventing, delaying, or reducing NODAT and/or PTMS associated complications including micro- and macrovascular diseases and events, graft rejection, infection, and death in a patient in need thereof characterized in that a DPP-4 inhibitor and, optionally, a second and, optionally, a third antidiabetic agent as defined hereinbefore and hereinafter are administered, for example in combination, to the patient.

According to another aspect of the invention, there is provided a method for treating hyperuricemia and hyperuricemia-associated conditions, such as for example gout, hypertension and renal failure, in a patient in need thereof characterized in that a DPP-4 inhibitor and, optionally, a second and, optionally, a third antidiabetic agent as defined hereinbefore and hereinafter are administered, for example in combination, to the patient.

According to another aspect of the invention there is provided the use of a DPP-4 inhibitor for the manufacture of a medicament for
preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity and metabolic syndrome; or
improving glycemic control and/or for reducing of fasting plasma glucose, of postprandial plasma glucose and/or of glycosylated hemoglobin HbA1c; or
preventing, slowing, delaying or reversing progression from impaired glucose tolerance (IGT), impaired fasting blood glucose (IFG), insulin resistance and/or from metabolic syndrome to type 2 diabetes mellitus; or
preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischaemia, arteriosclerosis, myocardial infarction, stroke and peripheral arterial occlusive disease; or
reducing body weight and/or body fat or preventing an increase in body weight and/or body fat or facilitating a reduction in body weight and/or body fat; or
preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion; or
preventing, slowing, delaying or treating diseases or conditions attributed to an abnormal accumulation of liver or ectopic fat; or
maintaining and/or improving the insulin sensitivity and/or for treating or preventing hyperinsulinemia and/or insulin resistance; or
for preventing, slowing progression of, delaying, or treating new onset diabetes after transplantation (NODAT) and/or post-transplant metabolic syndrome (PTMS); or
for preventing, delaying, or reducing NODAT and/or PTMS associated complications including micro- and macrovascular diseases and events, graft rejection, infection, and death; or
for treating hyperuricemia and hyperuricemia associated conditions;

in a patient in need thereof, optionally, characterized in that the DPP-4 inhibitor is administered, for example alone or in combination, with a second and, optionally, with a third antidiabetic agent as defined hereinbefore and hereinafter.

According to another aspect of the invention, there is provided the use of a second antidiabetic agent as defined hereinbefore and hereinafter for the manufacture of a medicament for
preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity and metabolic syndrome; or
improving glycemic control and/or for reducing of fasting plasma glucose, of postprandial plasma glucose and/or of glycosylated hemoglobin HbA1c; or
preventing, slowing, delaying or reversing progression from impaired glucose tolerance (IGT), impaired fasting blood glucose (IFG), insulin resistance and/or from metabolic syndrome to type 2 diabetes mellitus; or
preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischaemia, arteriosclerosis, myocardial infarction, stroke and peripheral arterial occlusive disease; or
reducing body weight and/or body fat or preventing an increase in body weight and/or body fat or facilitating a reduction in body weight and/or body fat; or
preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion; or
preventing, slowing, delaying or treating diseases or conditions attributed to an abnormal accumulation of liver or ectopic fat; or
maintaining and/or improving the insulin sensitivity and/or for treating or preventing hyperinsulinemia and/or insulin resistance;

in a patient in need thereof characterized in that the second antidiabetic agent is administered, for example in combination, with a DPP-4 inhibitor and, optionally, with a third antidiabetic agent as defined hereinbefore and hereinafter.

According to another aspect of the invention, there is provided the use of a pharmaceutical composition according to the present invention for the manufacture of a medicament for a therapeutic and preventive method as described hereinbefore and hereinafter.

### Definitions

The term “active ingredient” of a pharmaceutical composition according to the present invention means the DPP-4 inhibitor and/or the second antidiabetic agent and/or the third antidiabetic agent according to the present invention.

The term “body mass index” or “BMI” of a human patient is defined as the weight in kilograms divided by the square of the height in meters, such that BMI has units of kg/m².

The term “overweight” is defined as the condition wherein the individual has a BMI greater than or 25 kg/m² and less than 30 kg/m². The terms “overweight” and “pre-obese” are used interchangeably.

The term “obesity” is defined as the condition wherein the individual has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the term “class III obesity” is the condition wherein the BMI is equal to or greater than 40 kg/m².

The term “visceral obesity” is defined as the condition wherein a waist-to-hip ratio of greater than or equal to 1.0 in men and 0.8 in women is measured. It defines the risk for insulin resistance and the development of pre-diabetes.

The term “abdominal obesity” is usually defined as the condition wherein the waist circumference is >40 inches or 102 cm in men, and is >35 inches or 94 cm in women. With regard to a Japanese ethnicity or Japanese patients abdominal obesity may be defined as waist circumference ≥85 cm in men and >90 cm in women (see e.g. investigating committee for the diagnosis of metabolic syndrome in Japan).

The term “euglycemia” is defined as the condition in which a subject has a fasting blood glucose concentration within the normal range, greater than 70 mg/dL (3.89 mmol/L) and less than 110 mg/dL (6.11 mmol/L) or 100 mg mg/dL (5.6 mmol/L). The word “fasting” has the usual meaning as a medical term.

The term “hyperglycemia” is defined as the condition in which a subject has a fasting blood glucose concentration above the normal range, greater than 110 mg/dL (6.11 mmol/L) or 100 mg mg/dL (5.6 mmol/L). The word “fasting” has the usual meaning as a medical term.

The term “hypoglycemia” is defined as the condition in which a subject has a blood glucose concentration below the normal range of 60 to 115 mg/dL (3.3 to 6.3 mmol/L), in particular below 70 mg/dL (3.89 mmol/L).

The term “postprandial hyperglycemia” is defined as the condition in which a subject has a 2 hour postprandial blood glucose or serum glucose concentration greater than 200 mg/dL (11.11 mmol/L).

The term “impaired fasting blood glucose” or “IFG” is defined as the condition in which a subject has a fasting blood glucose concentration or fasting serum glucose concentration in a range from 100 to 125 mg/dl (i.e. from 5.6 to 6.9 mmol/I), in particular greater than 110 mg/dL and less than 126 mg/dl (7.00 mmol/L). A subject with “normal fasting glucose” has a fasting glucose concentration smaller than 100 mg/dl, i.e. smaller than 5.6 mmol/l.

The term “impaired glucose tolerance” or “IGT” is defined as the condition in which a subject has a 2 hour postprandial blood glucose or serum glucose concentration greater than 140 mg/dl (7.78 mmol/L) and less than 200 mg/dL (11.11 mmol/L). The abnormal glucose tolerance, i.e. the 2 hour postprandial blood glucose or serum glucose concentration can be measured as the blood sugar level in mg of glucose per dL of plasma 2 hours after taking 75 g of glucose after a fast. A subject with “normal glucose tolerance” has a 2 hour postprandial blood glucose or serum glucose concentration smaller than 140 mg/dl (7.78 mmol/L).

The term “hyperinsulinemia” is defined as the condition in which a subject with insulin resistance, with or without euglycemia, has fasting or postprandial serum or plasma insulin concentration elevated above that of normal, lean individuals without insulin resistance, having a waist-to-hip ratio <1.0 (for men) or <0.8 (for women).

The terms “insulin-sensitizing”, “insulin resistance-improving” or “insulin resistance-lowering” are synonymous and used interchangeably.

The term “insulin resistance” is defined as a state in which circulating insulin levels in excess of the normal response to a glucose load are required to maintain the euglycemic state (Ford E S, et al. JAMA. (2002) 287:356-9). A method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. With this method, it is not possible to distinguish between hepatic and peripheral insulin resistance.

Furthermore, insulin resistance, the response of a patient with insulin resistance to therapy, insulin sensitivity and hyperinsulinemia may be quantified by assessing the “homeostasis model assessment to insulin resistance (HOMA-IR)” score, a reliable indicator of insulin resistance (Katsuki A, et al. Diabetes Care 2001; 24: 362-5). Further reference is made to methods for the determination of the HOMA-index for insulin sensitivity (Matthews et al., Diabetologia 1985, 28: 412-19), of the ratio of intact proinsulin to insulin (Forst et al., Diabetes 2003, 52 (Suppl. 1): A459) and to an euglycemic clamp study. In addition, plasma adiponectin levels can be monitored as a potential surrogate of insulin sensitivity. The estimate of insulin resistance by the homeostasis assessment model (HOMA)-IR score is calculated with the formula (Galvin P, et al. Diabet Med 1992; 9:921-8):

HOMA-IR=[fasting serum insulin (μU/mL)]×[fasting plasma glucose(mmol/L)/22.5]

As a rule, other parameters are used in everyday clinical practice to assess insulin resistance. Preferably, the patient's triglyceride concentration is used, for example, as increased triglyceride levels correlate significantly with the presence of insulin resistance.

Patients with a predisposition for the development of IGT or IFG or type 2 diabetes are those having euglycemia with hyperinsulinemia and are by definition, insulin resistant. A typical patient with insulin resistance is usually overweight or obese. If insulin resistance can be detected, this is a particularly strong indication of the presence of pre-diabetes. Thus, it may be that in order to maintain glucose homoeostasis a person needs 2-3 times as much insulin as a healthy person, without this resulting in any clinical symptoms.

The methods to investigate the function of pancreatic beta-cells are similar to the above methods with regard to insulin sensitivity, hyperinsulinemia or insulin resistance: An improvement of beta-cell function can be measured for example by determining a HOMA-index for beta-cell function (Matthews et al., Diabetologia 1985, 28: 412-19), the ratio of intact proinsulin to insulin (Forst et al., Diabetes 2003, 52 (Suppl. 1): A459), the insulin/C-peptide secretion after an oral glucose tolerance test or a meal tolerance test, or by employing a hyperglycemic clamp study and/or minimal modeling after a frequently sampled intravenous glucose tolerance test (Stumvoll et al., Eur J Clin Invest 2001, 31: 380-81).

The term “pre-diabetes” is the condition wherein an individual is pre-disposed to the development of type 2 diabetes. Pre-diabetes extends the definition of impaired glucose tolerance to include individuals with a fasting blood glucose within the high normal range ≥100 mg/dL (J. B. Meigs, et al. Diabetes 2003; 52:1475-1484) and fasting hyperinsulinemia (elevated plasma insulin concentration). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749).

Individuals likely to have insulin resistance are those who have two or more of the following attributes: 1) overweight or obese, 2) high blood pressure, 3) hyperlipidemia, 4) one or more 1^{st} degree relative with a diagnosis of IGT or IFG or type 2 diabetes. Insulin resistance can be confirmed in these individuals by calculating the HOMA-IR score. For the purpose of this invention, insulin resistance is defined as the clinical condition in which an individual has a HOMA-IR score >4.0 or a HOMA-IR score above the upper limit of normal as defined for the laboratory performing the glucose and insulin assays.

The term “type 2 diabetes” is defined as the condition in which a subject has a fasting blood glucose or serum glucose concentration greater than 125 mg/dL (6.94 mmol/L). The measurement of blood glucose values is a standard procedure in routine medical analysis. If a glucose tolerance test is carried out, the blood sugar level of a diabetic will be in excess of 200 mg of glucose per dL (11.1 mmol/I) of plasma 2 hours after 75 g of glucose have been taken on an empty stomach. In a glucose tolerance test 75 g of glucose are administered orally to the patient being tested after 10-12 hours of fasting and the blood sugar level is recorded immediately before taking the glucose and 1 and 2 hours after taking it. In a healthy subject, the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg, this is regarded as abnormal glucose tolerance.

The term “late stage type 2 diabetes mellitus” includes patients (with type 2 diabetes) with a secondary (antidiabetic) drug failure, indication for insulin therapy and progression to micro- and macrovascular complications e.g. diabetic nephropathy, or coronary heart disease (CHD).

The term “HbA1c” refers to the product of a non-enzymatic glycation of the haemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5% of the total haemoglobin in the sample), are significantly better protected against diabetic microangiopathy. For example, metformin on its own achieves an average improvement in the HbA1c value in the diabetic of the order of 1.0-1.5%. This reduction of the HbA1C value is not sufficient in all diabetics to achieve the desired target range of <6.5% and preferably <6% HbA1c.

The term “insufficient glycemic control” or “inadequate glycemic control” in the scope of the present invention means a condition wherein patients show HbA1c values above 6.5%, in particular above 7.0%, even more preferably above 7.5%, especially above 8%.

The “metabolic syndrome”, also called “syndrome X” (when used in the context of a metabolic disorder), also called the “dysmetabolic syndrome” is a syndrome complex with the cardinal feature being insulin resistance (Laaksonen D E, et al. Am J Epidemiol 2002; 156:1070-7). According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association (2001) 285:2486-2497), diagnosis of the metabolic syndrome is made when three or more of the following risk factors are present:
1. Abdominal obesity, defined as waist circumference >40 inches or 102 cm in men, and >35 inches or 94 cm in women; or with regard to a Japanese ethnicity or Japanese patients defined as waist circumference ≥85 cm in men and >90 cm in women;
2. Triglycerides: >150 mg/dL
3. HDL-cholesterol <40 mg/dL in men
4. Blood pressure ≥130/85 mm Hg (SBP >130 or DBP >85)
5. Fasting blood glucose >110 mg/dL or >100 mg/dL

The NCEP definitions have been validated (Laaksonen D E, et al. Am J Epidemiol. (2002) 156:1070-7). Triglycerides and HDL cholesterol in the blood can also be determined by standard methods in medical analysis and are described for example in Thomas L (Editor): “Labor und Diagnose”, TH-Books Verlagsgesellschaft mbH, Frankfurt/Main, 2000.

According to a commonly used definition, hypertension is diagnosed if the systolic blood pressure (SBP) exceeds a value of 140 mm Hg and diastolic blood pressure (DBP) exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg.

The definitions of NODAT (new onset diabetes after transplantation) and PTMS (post-transplant metabolic syndrome) follow closely that of the American Diabetes Association diagnostic criteria for type 2 diabetes, and that of the International Diabetes Federation (IDF) and the American Heart Association/National Heart, Lung, and Blood Institute, for the metabolic syndrome. NODAT and/or PTMS are associated with an increased risk of micro- and macrovascular disease and events, graft rejection, infection, and death. A number of predictors have been identified as potential risk factors related to NODAT and/or PTMS including a higher age at transplant, male gender, the pre-transplant body mass index, pre-transplant diabetes, and immunosuppression.

The term “hyperuricemia” denotes a condition of high serum total urate levels. In human blood, uric acid concentrations between 3.6 mg/dL (ca. 214 μmol/L) and 8.3 mg/dL (ca. 494 μmol/L) are considered normal by the American Medical Association. High serum total urate levels, or hyperuricemia, are often associated with several maladies. For example, high serum total urate levels can lead to a type of arthritis in the joints known as gout. Gout is a condition created by a build up of monosodium urate or uric acid crystals on the articular cartilage of joints, tendons and surrounding tissues due to elevated concentrations of total urate levels in the blood stream. The build up of urate or uric acid on these tissues provokes an inflammatory reaction of these tissues. Saturation levels of uric acid in urine may result in kidney stone formation when the uric acid or urate crystallizes in the kidney. Additionally, high serum total urate levels are often associated with the so-called metabolic syndrome, including cardiovascular disease and hypertension.

The term “DPP-4 inhibitor” in the scope of the present invention relates to a compound that exhibits inhibitory activity on the enzyme dipeptidyl peptidase IV (DPP-4). Such inhibitory activity can be characterised by the IC50 value. A DPP-4 inhibitor preferably exhibits an IC50 value below 10000 nM, preferably below 1000 nM. Certain DPP-4 inhibitors exhibit an IC50 value below 100 nM, or even <50 nM. IC50 values of DPP-4 inhibitors are usually above 0.01 nM, or even above 0.1 nM. DPP-IV inhibitors may include biologic and non-biologic, in particular non-peptidic compounds. The inhibitory effect on DPP-4 can be determined by methods known in the literature, in particular as described in the application WO 02/068420 or WO 2004/018468 (page 34), which are incorporated herein by reference in its entirety.

The term “DPP-4 inhibitor” also comprises any pharmaceutically acceptable salts thereof, hydrates and solvates thereof, including the respective crystalline forms.

The terms “treatment” and “treating” comprise therapeutic treatment of patients having already developed said condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods of the present invention may be used for instance as therapeutic treatment over a period of time as well as for chronic therapy.

The terms “prophylactically treating”, “preventivally treating” and “preventing” are used interchangeably and comprise a treatment of patients at risk to develop a condition mentioned hereinbefore, thus reducing said risk.

### DETAILED DESCRIPTION

The aspects according to the present invention, in particular the pharmaceutical compositions, methods and uses, refer to DPP-4 inhibitors, second and/or third antidiabetic agents as defined hereinbefore and hereinafter. In the methods and uses according to this invention a second and, optionally, third antidiabetic agent may be optionally administered, i.e. the DPP-4 inhibitor is administered in combination with the second and, optionally, third antidiabetic agent or without a second and, optionally, third antidiabetic agent. In the methods and uses according to this invention a third antidiabetic agent may be optionally administered, i.e. the DPP-4 inhibitor and the second antidiabetic agent are administered in combination with a third antidiabetic agent or without a third antidiabetic agent.

In a first embodiment (embodiment A), a DPP-4 inhibitor in the context of the present invention is any DPP-4 inhibitor of

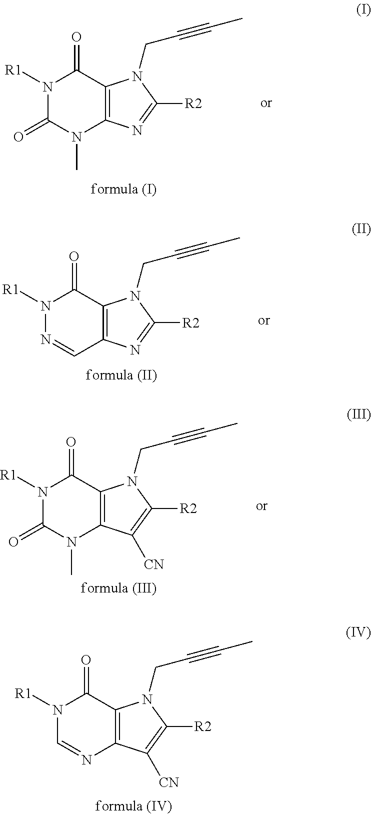

wherein R1 denotes ([1,5]naphthyridin-2-yl)methyl, (quinazolin-2-yl)methyl, (quinoxalin-6-yl)methyl, (4-methyl-quinazolin-2-yl)methyl, 2-cyano-benzyl, (3-cyano-quinolin-2-yl)methyl, (3-cyano-pyridin-2-yl)methyl, (4-methyl-pyrimidin-2-yl)methyl, or (4,6-dimethyl-pyrimidin-2-yl)methyl and R2 denotes 3-(R)-amino-piperidin-1-yl, (2-amino-2-methyl-propyl)-methylamino or (2-(S)-amino-propyl)-methylamino, or its pharmaceutically acceptable salt.

In a second embodiment (embodiment B), a DPP-4 inhibitor in the context of the present invention is a DPP-4 inhibitor selected from the group consisting of sitagliptin, vildagliptin, saxagliptin, alogliptin,
(2S)-1-{[2-(5-Methyl-2-phenyl-oxazol-4-yl)-ethylamino]-acetyl}-pyrrolidine-2-carbonitrile,
(2S)-1-{[1,1-Dimethyl-3-(4-pyridin-3-yl-imidazol-1-yl)-propylamino]-acetyl}-pyrrolidine-2-carbonitrile,
(S)-1-((2S,3S,11bS)-2-Amino-9,10-dimethoxy-1,3,4,6,7,11b-hexahydro-2H-pyrido[2,1-a]isoquinolin-3-yl)-4-fluoromethyl-pyrrolidin-2-one,
(3,3-Difluoropyrrolidin-1-yl)-((2S,4S)-4-(4-(pyrimidin-2-yl)piperazin-1-yl)pyrrolidin-2-yl)methanone,
(1 ((3S,4S)-4-amino-1-(4-(3,3-difluoropyrrolidin-1-yl)-1,3,5-triazin-2-yl)pyrrolidin-3-yl)-5,5-difluoropiperidin-2-one,
(2S,4S)-1-{2-[(3S,1R)-3-(1H-1,2,4-Triazol-1-ylmethyl)cyclopentylamino]-acetyl}-4-fluoropyrrolidine-2-carbonitrile,
(R)-2-[6-(3-Amino-piperidin-1-yl)-3-methyl-2,4-dioxo-3,4-dihydro-2H-pyrimidin-1-ylmethyl]-4-fluoro-benzonitrile,
5-{(S)-2-[2-((S)-2-Cyano-pyrrolidin-1-yl)-2-oxo-ethylamino]-propyl}-5-(1H-tetrazol-5-yl)-10,11-dihydro-5H-dibenzo[a,d]cycloheptene-2,8-dicarboxylic acid bis-dimethylamide,
3-{(2S,4S)-4-[4-(3-Methyl-1-phenyl-1H-pyrazol-5-yl)piperazin-1-yl]pyrrolidin-2-ylcarbonyl}thiazolidine,
[(2R)-1-{[(3R)-pyrrolidin-3-ylamino]acetyl}pyrrolidin-2-yl]boronic acid,
(2S,4S)-1-[2-[(4-ethoxycarbonylbicyclo[2.2.2]oct-1-yl)amino]acetyl]-4-fluoropyrrolidine-2-carbonitrile,
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile, and
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione,
or its pharmaceutically acceptable salt.

Regarding the first embodiment (embodiment A), preferred DPP-4 inhibitors are any or all of the following compounds and their pharmaceutically acceptable salts:
1-[(4-methyl-quinazolin-2-yl)methyl]-3-methyl-7-(2-butyn-1-yl)-8-(3-(R)-amino-piperidin-1-yl)-xanthine (compare WO 2004/018468, example 2(142)):

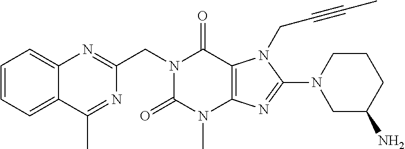 [SMILES: CC#CCn1c(N2CCC[C@@H](N)C2)nc2c1c(=O)n(Cc1nc(C)c3ccccc3n1)c(=O)n2C]

1-[([1,5]naphthyridin-2-yl)methyl]-3-methyl-7-(2-butyn-1-yl)-8-((R)-3-amino-piperidin-1-yl)-xanthine (compare WO 2004/018468, example 2(252)):

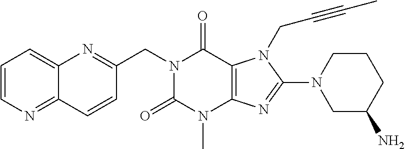 [SMILES: CC#CCn1c(N2CCC[C@@H](N)C2)nc2c1c(=O)n(Cc1ccc3ncccc3n1)c(=O)n2C]

1-[(Quinazolin-2-yl)methyl]-3-methyl-7-(2-butyn-1-yl)-8-((R)-3-amino-piperidin-1-yl)-xanthine (compare WO 2004/018468, example 2(80)):

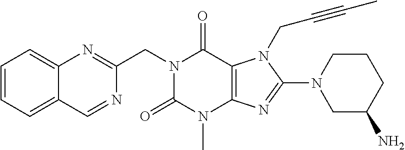 [SMILES: CC#CCn1c(N2CCC[C@@H](N)C2)nc2c1c(=O)n(Cc1ncc3ccccc3n1)c(=O)n2C]

2-((R)-3-Amino-piperidin-1-yl)-3-(but-2-yinyl)-5-(4-methyl-quinazolin-2-ylmethyl)-3,5-dihydro-imidazo[4,5-d]pyridazin-4-one (compare WO 2004/050658, example 136):

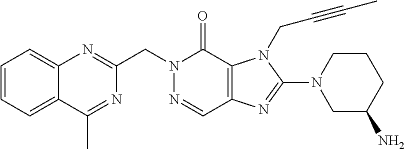 [SMILES: CC#CCn1c(N2CCC[C@@H](N)C2)nc2cnn(Cc3nc(C)c4ccccc4n3)c(=O)c21]

1-[(4-Methyl-quinazolin-2-yl)methyl]-3-methyl-7-(2-butyin-1-yl)-8-[(2-amino-2-methyl-propyl)-methylamino]-xanthine (compare WO 2006/029769, example 2(1)):

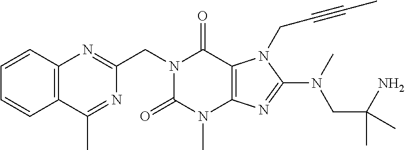 [SMILES: CC#CCn1c(N(C)CC(C)(C)N)nc2c1c(=O)n(Cc1nc(C)c3ccccc3n1)c(=O)n2C]

1-[(3-Cyano-quinolin-2-yl)methyl]-3-methyl-7-(2-butyn-1-yl)-8-((R)-3-amino-piperidin-1-yl)-xanthine (compare WO 2005/085246, example 1(30)):

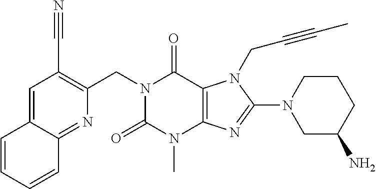 [SMILES: CC#CCn1c(N2CCC[C@@H](N)C2)nc2c1c(=O)n(Cc1nc3ccccc3cc1C#N)c(=O)n2C]

1-(2-Cyano-benzyl)-3-methyl-7-(2-butyn-1-yl)-8-((R)-3-amino-piperidin-1-yl)-xanthine (compare WO 2005/085246, example 1(39)):

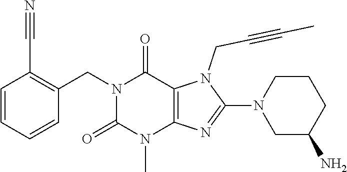 [SMILES: CC#CCn1c(N2CCC[C@@H](N)C2)nc2c1c(=O)n(Cc1ccccc1C#N)c(=O)n2C]

1-[(4-Methyl-quinazolin-2-yl)methyl]-3-methyl-7-(2-butyn-1-yl)-8-[(S)-(2-amino-propyl)-methylamino]-xanthine (compare WO 2006/029769, example 2(4)):

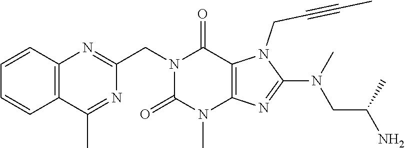 [SMILES: CC#CCn1c(N(C)C[C@H](C)N)nc2c1c(=O)n(Cc1nc(C)c3ccccc3n1)c(=O)n2C]

1-[(3-Cyano-pyridin-2-yl)methyl]-3-methyl-7-(2-butyn-1-yl)-8-((R)-3-amino-piperidin-1-yl)-xanthine (compare WO 2005/085246, example 1(52)):

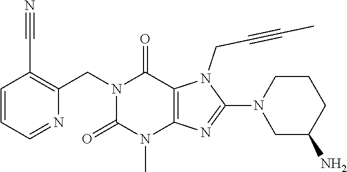 [SMILES: CC#CCn1c(N2CCC[C@@H](N)C2)nc2c1c(=O)n(Cc1ncccc1C#N)c(=O)n2C]

1-[(4-Methyl-pyrimidin-2-yl)methyl]-3-methyl-7-(2-butyn-1-yl)-8-((R)-3-amino-piperidin-1-yl)-xanthine (compare WO 2005/085246, example 1(81)):

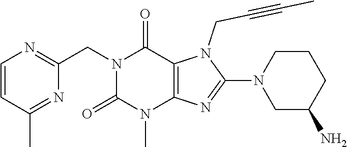 [SMILES: CC#CCn1c(N2CCC[C@@H](N)C2)nc2c1c(=O)n(Cc1nccc(C)n1)c(=O)n2C]

1-[(4,6-Dimethyl-pyrimidin-2-yl)methyl]-3-methyl-7-(2-butyn-1-yl)-8-((R)-3-amino-piperidin-1-yl)-xanthine (compare WO 2005/085246, example 1(82)):

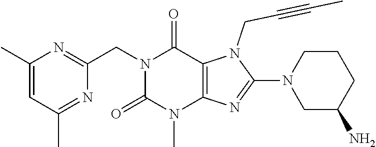 [SMILES: CC#CCn1c(N2CCC[C@@H](N)C2)nc2c1c(=O)n(Cc1nc(C)cc(C)n1)c(=O)n2C]

1-[(Quinoxalin-6-yl)methyl]-3-methyl-7-(2-butyn-1-yl)-8-((R)-3-amino-piperidin-1-yl)-xanthine (compare WO 2005/085246, example 1(83)):

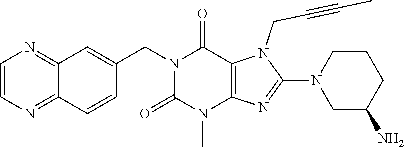 [SMILES: CC#CCn1c(N2CCC[C@@H](N)C2)nc2c1c(=O)n(Cc1ccc3nccnc3c1)c(=O)n2C]

A more preferred DPP-4 inhibitor among the abovementioned DPP-4 inhibitors of embodiment A of this invention is 1-[(4-methyl-quinazolin-2-yl)methyl]-3-methyl-7-(2-butyn-1-yl)-8-(3-(R)-amino-piperidin-1-yl)-xanthine, particularly the free base thereof (which is also known as linagliptin or BI 1356).

As further DPP-4 inhibitors the following compounds can be mentioned:
Sitagliptin (MK-0431) having the structural formula A below is (3R)-3-amino-1-[3-(trifluoromethyl)-5,6,7,8-tetrahydro-5H-[1,2,4]triazolo[4,3-a]pyrazin-7-yl]-4-(2,4,5-trifluorophenyl)butan-1-one, also named (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine,

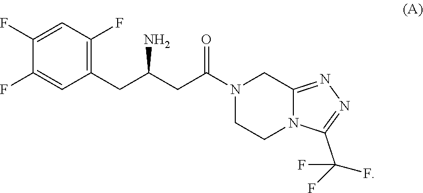 [SMILES: N[C@@H](CC(=O)N1CCn2c(nnc2C(F)(F)F)C1)Cc1cc(F)c(F)cc1F]

In one embodiment, sitagliptin is in the form of its dihydrogenphosphate salt, i.e. sitagliptin phosphate. In a further embodiment, sitagliptin phosphate is in the form of a crystalline anhydrate or monohydrate. A class of this embodiment refers to sitagliptin phosphate monohydrate. Sitagliptin free base and pharmaceutically acceptable salts thereof are disclosed in U.S. Pat. No. 6,699,871 and in Example 7 of WO 03/004498. Crystalline sitagliptin phosphate monohydrate is disclosed in WO 2005/003135 and in WO 2007/050485.

For details, e.g. on a process to manufacture, to formulate or to use this compound or a salt thereof, reference is thus made to these documents.

A tablet formulation for sitagliptin is commercially available under the trade name Januvia®. A tablet formulation for sitagliptin/metformin combination is commercially available under the trade name Janumet®.
Vildagliptin (LAF-237) having the structural formula B below is (2S)-{[(3-hydroxyadamantan-1-yl)amino]acetyl}pyrrolidine-2-carbonitrile, also named (S)-1-[(3-hydroxy-1-adamantyl)amino]acetyl-2-cyano-pyrrolidine,

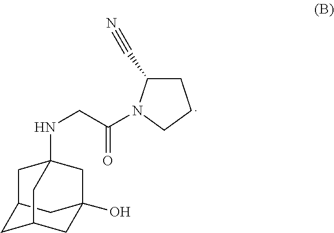 [SMILES: N#C[C@@H]1CCCN1C(=O)CNC12CC3CC(CC(O)(C3)C1)C2]

Vildagliptin is specifically disclosed in U.S. Pat. No. 6,166,063 and in Example 1 of WO 00/34241. Specific salts of vildagliptin are disclosed in WO 2007/019255. A crystalline form of vildagliptin as well as a vildagliptin tablet formulation are disclosed in WO 2006/078593. Vildagliptin can be formulated as described in WO 00/34241 or in WO 2005/067976. A modified release vildagliptin formulation is described in WO 2006/135723.

For details, e.g. on a process to manufacture, to formulate or to use this compound or a salt thereof, reference is thus made to these documents.

A tablet formulation for vildagliptin is expected to be commercially available under the trade name Galvus®. A tablet formulation for vildagliptin/metformin combination is commercially available under the trade name Eucreas®.
Saxagliptin (BMS-477118) having the structural formula C below is (1S,3S,5S)-2-{(2S)-2-amino-2-(3-hydroxyadamantan-1-yl)acetyl}-2-azabicyclo[3.1.0]hexane-3-carbonitrile, also named (S)-3-hydroxyadamantylglycine-L-cis-4,5-methanoprolinenitrile,

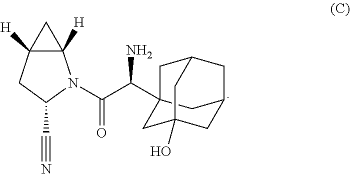 [SMILES: N#C[C@@H]1C[C@@H]2C[C@@H]2N1C(=O)[C@@H](N)C12CC3CC(CC(O)(C3)C1)C2]

Saxagliptin is specifically disclosed in U.S. Pat. No. 6,395,767 and in Example 60 of WO 01/68603.

In one embodiment, saxagliptin is in the form of its HCl salt or its mono-benzoate salt as disclosed in WO 2004/052850. In a further embodiment, saxagliptin is in the form of the free base. In a yet further embodiment, saxagliptin is in the form of the monohydrate of the free base as disclosed in WO 2004/052850. Crystalline forms of the HCl salt and of the free base of saxagliptin are disclosed in WO 2008/131149. A process for preparing saxagliptin is also disclosed in WO 2005/106011 and WO 2005/115982. Saxagliptin can be formulated in a tablet as described in WO 2005/117841.

For details, e.g. on a process to manufacture, to formulate or to use this compound or a salt thereof, reference is thus made to these documents.
Alogliptin (SYR-322) having the structural formula E below is 2-({6-[(3R)-3-aminopiperidin-1-yl]-3-methyl-2,4-dioxo-3,4-dihydro-2H-pyrimidin-1-yl}methyl)benzonitrile

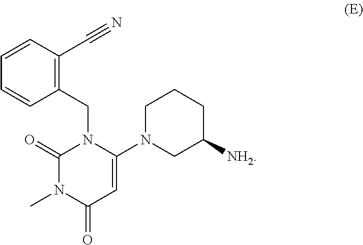 [SMILES: Cn1c(=O)cc(N2CCC[C@@H](N)C2)n(Cc2ccccc2C#N)c1=O]

Alogliptin is specifically disclosed in US 2005/261271, EP 1586571 and in WO 2005/095381. In one embodiment, alogliptin is in the form of its benzoate salt, its hydrochloride salt or its tosylate salt each as disclosed in WO 2007/035629. A class of this embodiment refers to alogliptin benzoate. Polymorphs of alogliptin benzoate are disclosed in WO 2007/035372. A process for preparing alogliptin is disclosed in WO 2007/112368 and, specifically, in WO 2007/035629. Alogliptin (namely its benzoate salt) can be formulated in a tablet and administered as described in WO 2007/033266. A solid preparation of alogliptin/pioglitazone and its preparation and use is described in WO 2008/093882. A solid preparation of alogliptin/metformin and its preparation and use is described in WO 2009/011451.

For details, e.g. on a process to manufacture, to formulate or to use this compound or a salt thereof, reference is thus made to these documents.
(2S)-1-{[2-(5-Methyl-2-phenyl-oxazol-4-yl)-ethylamino]-acetyl}-pyrrolidine-2-carbonitrile or a pharmaceutically acceptable salt thereof, preferably the mesylate, or
(2S)-1-{[1,1-Dimethyl-3-(4-pyridin-3-yl-imidazol-1-yl)-propylamino]-acetyl}-pyrrolidine-2-carbonitrile or a pharmaceutically acceptable salt thereof:

These compounds and methods for their preparation are disclosed in WO 03/037327. The mesylate salt of the former compound as well as crystalline polymorphs thereof are disclosed in WO 2006/100181. The fumarate salt of the latter compound as well as crystalline polymorphs thereof are disclosed in WO 2007/071576. These compounds can be formulated in a pharmaceutical composition as described in WO 2007/017423.

For details, e.g. on a process to manufacture, to formulate or to use these compounds or salts thereof, reference is thus made to these documents.
(S)-1-((2S,3S,11bS)-2-Amino-9,10-dimethoxy-1,3,4,6,7,11b-hexahydro-2H-pyrido[2,1-a]isoquinolin-3-yl)-4-fluoromethyl-pyrrolidin-2-one (also named carmegliptin) or a pharmaceutically acceptable salt thereof:

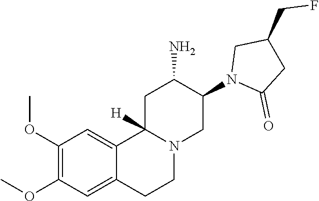 [SMILES: COc1cc2c(cc1OC)[C@@H]1C[C@H](N)[C@@H](N3C[C@@H](CF)CC3=O)CN1CC2]

This compound and methods for its preparation are disclosed in WO 2005/000848. A process for preparing this compound (specifically its dihydrochloride salt) is also disclosed in WO 2008/031749, WO 2008/031750 and WO 2008/055814. This compound can be formulated in a pharmaceutical composition as described in WO 2007/017423.

For details, e.g. on a process to manufacture, to formulate or to use this compound or a salt thereof, reference is thus made to these documents.
(3,3-Difluoropyrrolidin-1-yl)-((2S,4S)-4-(4-(pyrimidin-2-yl)piperazin-1-yl)pyrrolidin-2-yl)methanone (also named gosogliptin) or a pharmaceutically acceptable salt thereof:

This compound and methods for its preparation are disclosed in WO 2005/116014 and U.S. Pat. No. 7,291,618.

For details, e.g. on a process to manufacture, to formulate or to use this compound or a salt thereof, reference is thus made to these documents.
(1 ((3S,4S)-4-amino-1-(4-(3,3-difluoropyrrolidin-1-yl)-1,3,5-triazin-2-yl)pyrrolidin-3-yl)-5,5-difluoropiperidin-2-one or a pharmaceutically acceptable salt thereof:

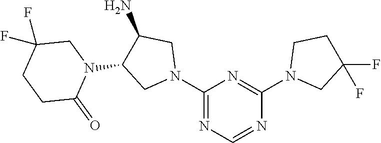 [SMILES: N[C@H]1CN(c2ncnc(N3CCC(F)(F)C3)n2)C[C@@H]1N1CC(F)(F)CCC1=O]

This compound and methods for its preparation are disclosed in WO 2007/148185 and US 20070299076. For details, e.g. on a process to manufacture, to formulate or to use this compound or a salt thereof, reference is thus made to these documents.
(2S,4S)-1-{2-[(3S,1R)-3-(1H-1,2,4-Triazol-1-ylmethyl)cyclopentylamino]-acetyl}-4-fluoropyrrolidine-2-carbonitrile (also named melogliptin) or a pharmaceutically acceptable salt thereof:

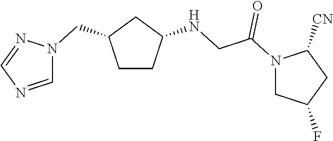 [SMILES: N#C[C@@H]1C[C@H](F)CN1C(=O)CN[C@@H]1CC[C@H](Cn2cncn2)C1]

This compound and methods for its preparation are disclosed in WO 2006/040625 and WO 2008/001195. Specifically claimed salts include the methanesulfonate and p-toluenesulfonate. For details, e.g. on a process to manufacture, to formulate or to use this compound or a salt thereof, reference is thus made to these documents.
(R)-2-[6-(3-Amino-piperidin-1-yl)-3-methyl-2,4-dioxo-3,4-dihydro-2H-pyrimidin-1-ylmethyl]-4-fluoro-benzonitrile or a pharmaceutically acceptable salt thereof:

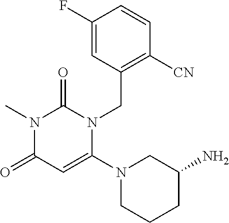 [SMILES: Cn1c(=O)cc(N2CCC[C@@H](N)C2)n(Cc2cc(F)ccc2C#N)c1=O]

This compound and methods for its preparation and use are disclosed in WO 2005/095381, US 2007060530, WO 2007/033350, WO 2007/035629, WO 2007/074884, WO 2007/112368, WO 2008/033851, WO 2008/114800 and WO 2008/114807. Specifically claimed salts include the succinate (WO 2008/067465), benzoate, benzenesulfonate, p-toluenesulfonate, (R)-mandelate and hydrochloride. For details, e.g. on a process to manufacture, to formulate or to use this compound or a salt thereof, reference is thus made to these documents.
5-{(S)-2-[2-((S)-2-Cyano-pyrrolidin-1-yl)-2-oxo-ethylamino]-propyl}-5-(1H-tetrazol-5-yl)-10,11-dihydro-5H-dibenzo[a,d]cycloheptene-2,8-dicarboxylic acid bis-dimethylamide or a pharmaceutically acceptable salt thereof:

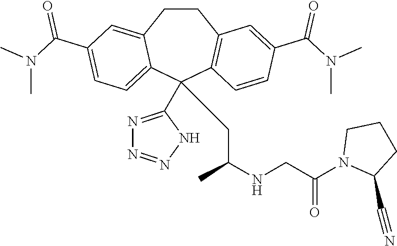 [SMILES: C[C@@H](CC1(c2nnn[nH]2)c2ccc(C(=O)N(C)C)cc2CCc2cc(C(=O)N(C)C)ccc21)NCC(=O)N1CCC[C@H]1C#N]

This compound and methods for its preparation are disclosed in WO 2006/116157 and US 2006/270701. For details, e.g. on a process to manufacture, to formulate or to use this compound or a salt thereof, reference is thus made to these documents.
3-{(2S,4S)-4-[4-(3-Methyl-1-phenyl-1H-pyrazol-5-yl)piperazin-1-yl]pyrrolidin-2-ylcarbonyl}thiazolidine (also named teneligliptin) or a pharmaceutically acceptable salt thereof:

This compound and methods for its preparation are disclosed in WO 02/14271. Specific salts are disclosed in WO 2006/088129 and WO 2006/118127 (including hydrochloride, hydrobromide, inter alia). Combination therapy using this compound is described in WO 2006/129785. For details, e.g. on a process to manufacture, to formulate or to use this compound or a salt thereof, reference is thus made to these documents.
[(2R)-1-{[(3R)-pyrrolidin-3-ylamino]acetyl}pyrrolidin-2-yl]boronic acid (also named dutogliptin) or a pharmaceutically acceptable salt thereof:

This compound and methods for its preparation are disclosed in WO 2005/047297, WO 2008/109681 and WO 2009/009751. Specific salts are disclosed in WO 2008/027273 (including citrate, tartrate). A formulation of this compound is described in WO 2008/144730. For details, e.g. on a process to manufacture, to formulate or to use this compound or a salt thereof, reference is thus made to these documents.
(2S,4S)-1-[2-[(4-ethoxycarbonylbicyclo[2.2.2]oct-1-yl)amino]acetyl]-4-fluoropyrrolidine-2-carbonitrile or a pharmaceutically acceptable salt thereof:

This compound and methods for its preparation are disclosed in WO 2005/075421, US 2008/146818 and WO 2008/114857. For details, e.g. on a process to manufacture, to formulate or to use this compound or a salt thereof, reference is thus made to these documents.
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile or a pharmaceutically acceptable salt thereof, or 6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione or a pharmaceutically acceptable salt thereof:

These compounds and methods for their preparation are disclosed in WO 2009/084497 and WO 2006/068163, respectively. For details, e.g. on a process to manufacture, to formulate or to use these compounds or salts thereof, reference is thus made to these documents.

Preferably the DPP-4 inhibitor is selected from the group G2 consisting of linagliptin, sitagliptin, vildagliptin, alogliptin, saxagliptin, carmegliptin, melogliptin, gosogliptin, teneligliptin and dutogliptin, or a pharmaceutically acceptable salt of one of the herein mentioned DPP-4 inhibitors, or a prodrug thereof.

A particularly preferred DPP-4 inhibitor to be emphasized within the present invention is linagliptin. The term “linagliptin” as employed herein refers to linagliptin and pharmaceutically acceptable salts thereof, including hydrates and solvates thereof, and crystalline forms thereof. Crystalline forms are described in WO 2007/128721. Methods for the manufacture of linagliptin are described in the patent applications WO 2004/018468 and WO 2006/048427 for example. Linagliptin is distinguished from structurally comparable DPP-4 inhibitors, as it combines exceptional potency and a long-lasting effect with favourable pharmacological properties, receptor selectivity and a favourable side-effect profile or bring about unexpected therapeutic advantages or improvements in monotherapy and/or when used in combination with a second and, optionally, a third antidiabetic agent according to this invention.

For avoidance of any doubt, the disclosure of each of the foregoing documents cited above in connection with the specified DPP-4 inhibitors is specifically incorporated herein by reference in its entirety.

In one aspect of the present invention, the pharmaceutical compositions, methods and uses according to this invention relate to those compositions which comprise the DPP-4 inhibitor as sole active ingredient (i.e. the second and third antidiabetic agent are both absent) and/or, respectively, to monotherapy using the DPP-4 inhibitor alone.

In another aspect of the present invention, the pharmaceutical compositions, combinations, methods and uses according to this invention relate to those compositions or combinations which comprise the DPP-4 inhibitor and the second antidiabetic agent as sole active ingredients (i.e. the third antidiabetic agent is absent) and/or, respectively, to dual combination therapy using the DPP-4 inhibitor and the second antidiabetic agent.

In another aspect of the present invention, the pharmaceutical compositions, combinations, methods and uses according to this invention relate to those compositions or combinations which comprise the DPP-4 inhibitor, the second and the third antidiabetic agent and/or, respectively, to triple combination therapy using the DPP-4 inhibitor, the second and the third antidiabetic agent.

Further, a DPP-4 inhibitor according to this invention may be further characterized in that said DPP-4 inhibitor does not significantly impair glomerular and/or tubular function of a type 2 diabetes patient with chronic renal insufficiency (e.g. mild, moderate or severe renal impairment or end stage renal disease), and/or

said DPP-4 inhibitor does not require to be dose-adjusted in a type 2 diabetes patient with impaired renal function (e.g. mild, moderate or severe renal impairment or end stage renal disease).

The second antidiabetic agent and, if present, the third antidiabetic agent is selected from the group G3 consisting of biguanides, thiazolidindiones, sulfonylureas, glinides, inhibitors of alpha-glucosidase, GLP-1 analogues or a pharmaceutically acceptable salt thereof. In the following preferred embodiments regarding the second and/or the third antidiabetic agent are described.

The group G3 comprises biguanides. Examples of biguanides are metformin, phenformin and buformin. A preferred biguanide is metformin. A DPP-4 inhibitor in combination with a biguanide, in particular metformin, can provide more efficacious glycemic control and/or may act together with the biguanide, for example to reduce weight, that has e.g. overall beneficial effects on the metabolic syndrome which is commonly associated with type 2 diabetes mellitus.

The term “metformin” as employed herein refers to metformin or a pharmaceutically acceptable salt thereof such as the hydrochloride salt, the metformin (2:1) fumarate salt, and the metformin (2:1) succinate salt, the hydrobromide salt, the p-chlorophenoxy acetate or the embonate, and other known metformin salts of mono and dibasic carboxylic acids. It is preferred that the metformin employed herein is the metformin hydrochloride salt.

The group G3 comprises thiazolidindiones. Examples of thiazolidindiones (TZD) are pioglitazone and rosiglitazone. TZD therapy is associated with weight gain and fat redistribution. In addition, TZD cause fluid retention and are not indicated in patients with congestive heart failure. Long term treatment with TZD are further associated with an increased risk of bone fractures. A DPP-4 inhibitor in combination with a thiazolidindione, in particular pioglitazone, can provide more efficacious glycemic control and/or can minimize side effects of the treatment with TZD.

The term “pioglitazone” as employed herein refers to pioglitazone, including its enantiomers, mixtures thereof and its racemate, or a pharmaceutically acceptable salt thereof such as the hydrochloride salt.

The term “rosiglitazone” as employed herein refers to rosiglitazone, including its enantiomers, mixtures thereof and its racemate, or a pharmaceutically acceptable salt thereof such as the maleate salt.

The group G3 comprises sulfonylureas. Examples of sulfonylureas are glibenclamide, tolbutamide, glimepiride, glipizide, gliquidone, glibornuride, glyburide, glisoxepide and gliclazide. Preferred sulfonylureas are tolbutamide, gliquidone, glibenclamide and glimepiride, in particular glibenclamide and glimepiride. As the efficacy of sulfonylureas wears off over the course of treatment, a combination of a DPP-4 inhibitor with a sulfonylurea may offer additional benefit to the patient in terms of better glycemic control. Also, treatment with sulfonylureas is normally associated with gradual weight gain over the course of treatment and a DPP-4 inhibitor may minimize this side effect of the treatment with an sulfonylurea and/or improve the metabolic syndrome. Also, a DPP-4 inhibitor in combination with a sulfonylurea may minimize hypoglycemia which is another undesirable side effect of sulfonylureas. This combination may also allow a reduction in the dose of sulfonylureas, which may also translate into less hypoglycemia.

Each term of the group “glibenclamide”, “glimepiride”, “gliquidone”, “glibornuride”, “gliclazide”, “glisoxepide”, “tolbutamide” and “glipizide” as employed herein refers to the respective active drug or a pharmaceutically acceptable salt thereof.

The group G3 comprises glinides. Examples of glinides are nateglinide, repaglinide and mitiglinide. As their efficacy wears off over the course of treatment, a combination of a DPP-4 inhibitor with a meglitinide may offer additional benefit to the patient in terms of better glycemic control. Also, treatment with meglitinides is normally associated with gradual weight gain over the course of treatment and a DPP-4 inhibitor may minimize this side effect of the treatment with an meglitinide and/or improve the metabolic syndrome. Also, a DPP-4 inhibitor in combination with a meglitinide may minimize hypoglycemia which is another undesirable side effect of meglitinides. This combination may also allow a reduction in the dose of meglitinides, which may also translate into less hypoglycemia.

The term “nateglinide” as employed herein refers to nateglinide, including its enantiomers, mixtures thereof and its racemate, or a pharmaceutically acceptable salts and esters thereof.

The term “repaglinide” as employed herein refers to repaglinide, including its enantiomers, mixtures thereof and its racemate, or a pharmaceutically acceptable salts and esters thereof.

The group G3 comprises inhibitors of alpha-glucosidase. Examples of inhibitors of alpha-glucosidase are acarbose, voglibose and miglitol. Additional benefits from the combination of a DPP-4 inhibitor and an alpha-glucosidase inhibitor may relate to more efficacious glycemic control, e.g. at lower doses of the individual drugs, and/or reducement of undesirable gastrointestinal side effects of alpha-glucosidase inhibitors.

Each term of the group “acarbose”, “voglibose” and “miglitol” as employed herein refers to the respective active drug or a pharmaceutically acceptable salt thereof.

The group G3 comprises inhibitors of GLP-1 analogues. Examples of GLP-1 analogues are exenatide, liraglutide, taspoglutide, semaglutide, albiglutide, and lixisenatide. The combination of a DPP-4 inhibitor and a GLP-1 analogue may achieve a superior glycemic control, e.g. at lower doses of the individual drugs. In addition, e.g. the body weight reducing capability of the GLP-1 analogue may be positively act together with the properties of the DPP-4 inhibitor. On the other hand, a reduction of side effects (e.g. nausea, gastrointestinal side effects like vomiting) may be obtained, e.g. when a reduced dose of the GLP-1 analogue is applied in the combination with a DPP-4 inhibitor.

Each term of the group “exenatide”, “liraglutide”, “taspoglutide”, “semaglutide”, “albiglutide” and “lixisenatide” as employed herein refers to the respective active drug or a pharmaceutically acceptable salt thereof.

In an embodiment (embodiment E1) the pharmaceutical compositions, combinations methods and uses according to this invention relate to combinations wherein the DPP-4 inhibitor and the second antidiabetic agent are preferably selected according to the entries in the Table 1.

TABLE 1

| DPP-4 Inhibitor | Second Antidiabetic Agent |
|---|---|
| selected from embodiment B | selected from the group G3 |
| selected from embodiment B | Metformin |
| selected from embodiment B | Pioglitazone |
| selected from embodiment B | Rosiglitazone |
| selected from embodiment B | Glibenclamide |
| selected from embodiment B | Glimepiride |
| selected from embodiment B | Gliquidone |
| selected from embodiment B | Nateglinide |
| selected from embodiment B | Repaglinide |
| selected from embodiment B | Acarbose |
| selected from embodiment B | Voglibose |
| selected from embodiment B | Miglitol |
| selected from embodiment B | Exenatide |
| selected from embodiment B | Liraglutide |
| selected from embodiment B | Taspoglutide |
| selected from embodiment B | Semaglutide |
| selected from embodiment B | Albiglutide |
| selected from embodiment B | Lixisenatide |
| Linagliptin | selected from the group G3 |
| Linagliptin | Metformin |
| Linagliptin | Pioglitazone |
| Linagliptin | Rosiglitazone |
| Linagliptin | Glibenclamide |
| Linagliptin | Glimepiride |
| Linagliptin | Gliquidone |
| Linagliptin | Nateglinide |
| Linagliptin | Repaglinide |
| Linagliptin | Acarbose |
| Linagliptin | Voglibose |
| Linagliptin | Miglitol |
| Linagliptin | Exenatide |
| Linagliptin | Liraglutide |
| Linagliptin | Taspoglutide |
| Linagliptin | Semaglutide |
| Linagliptin | Albiglutide |
| Linagliptin | Lixisenatide |
| Sitagliptin | selected from the group G3 |
| Sitagliptin | Metformin |
| Sitagliptin | Pioglitazone |
| Sitagliptin | Rosiglitazone |
| Sitagliptin | Glibenclamide |
| Sitagliptin | Glimepiride |
| Sitagliptin | Gliquidone |
| Sitagliptin | Nateglinide |
| Sitagliptin | Repaglinide |
| Sitagliptin | Acarbose |
| Sitagliptin | Voglibose |
| Sitagliptin | Miglitol |
| Sitagliptin | Exenatide |
| Sitagliptin | Liraglutide |
| Sitagliptin | Taspoglutide |
| Sitagliptin | Semaglutide |
| Sitagliptin | Albiglutide |
| Sitagliptin | Lixisenatide |
| Vildagliptin | selected from the group G3 |
| Vildagliptin | Metformin |
| Vildagliptin | Pioglitazone |
| Vildagliptin | Rosiglitazone |
| Vildagliptin | Glibenclamide |
| Vildagliptin | Glimepiride |
| Vildagliptin | Gliquidone |
| Vildagliptin | Nateglinide |
| Vildagliptin | Repaglinide |
| Vildagliptin | Acarbose |
| Vildagliptin | Voglibose |
| Vildagliptin | Miglitol |
| Vildagliptin | Exenatide |
| Vildagliptin | Liraglutide |
| Vildagliptin | Taspoglutide |
| Vildagliptin | Semaglutide |
| Vildagliptin | Albiglutide |
| Vildagliptin | Lixisenatide |
| Alogliptin | selected from the group G3 |
| Alogliptin | Metformin |
| Alogliptin | Pioglitazone |
| Alogliptin | Rosiglitazone |
| Alogliptin | Glibenclamide |
| Alogliptin | Glimepiride |
| Alogliptin | Gliquidone |
| Alogliptin | Nateglinide |
| Alogliptin | Repaglinide |
| Alogliptin | Acarbose |
| Alogliptin | Voglibose |
| Alogliptin | Miglitol |
| Alogliptin | Exenatide |
| Alogliptin | Liraglutide |
| Alogliptin | Taspoglutide |
| Alogliptin | Semaglutide |
| Alogliptin | Albiglutide |
| Alogliptin | Lixisenatide |
| Saxagliptin | selected from the group G3 |
| Saxagliptin | Metformin |
| Saxagliptin | Pioglitazone |
| Saxagliptin | Rosiglitazone |
| Saxagliptin | Glibenclamide |
| Saxagliptin | Glimepiride |
| Saxagliptin | Gliquidone |
| Saxagliptin | Nateglinide |
| Saxagliptin | Repaglinide |
| Saxagliptin | Acarbose |
| Saxagliptin | Voglibose |
| Saxagliptin | Miglitol |
| Saxagliptin | Exenatide |
| Saxagliptin | Liraglutide |
| Saxagliptin | Taspoglutide |
| Saxagliptin | Semaglutide |
| Saxagliptin | Albiglutide |
| Saxagliptin | Lixisenatide |
| Carmegliptin | selected from the group G3 |
| Carmegliptin | Metformin |
| Carmegliptin | Pioglitazone |
| Carmegliptin | Rosiglitazone |
| Carmegliptin | Glibenclamide |
| Carmegliptin | Glimepiride |
| Carmegliptin | Gliquidone |
| Carmegliptin | Nateglinide |
| Carmegliptin | Repaglinide |
| Carmegliptin | Acarbose |
| Carmegliptin | Voglibose |
| Carmegliptin | Miglitol |
| Carmegliptin | Exenatide |
| Carmegliptin | Liraglutide |
| Carmegliptin | Taspoglutide |
| Carmegliptin | Semaglutide |
| Carmegliptin | Albiglutide |
| Carmegliptin | Lixisenatide |
| Melogliptin | selected from the group G3 |
| Melogliptin | Metformin |
| Melogliptin | Pioglitazone |
| Melogliptin | Rosiglitazone |
| Melogliptin | Glibenclamide |
| Melogliptin | Glimepiride |
| Melogliptin | Gliquidone |
| Melogliptin | Nateglinide |
| Melogliptin | Repaglinide |
| Melogliptin | Acarbose |
| Melogliptin | Voglibose |
| Melogliptin | Miglitol |
| Melogliptin | Exenatide |
| Melogliptin | Liraglutide |
| Melogliptin | Taspoglutide |
| Melogliptin | Semaglutide |
| Melogliptin | Albiglutide |
| Melogliptin | Lixisenatide |
| Gosogliptin | selected from the group G3 |
| Gosogliptin | Metformin |
| Gosogliptin | Pioglitazone |
| Gosogliptin | Rosiglitazone |
| Gosogliptin | Glibenclamide |
| Gosogliptin | Glimepiride |
| Gosogliptin | Gliquidone |
| Gosogliptin | Nateglinide |
| Gosogliptin | Repaglinide |
| Gosogliptin | Acarbose |
| Gosogliptin | Voglibose |
| Gosogliptin | Miglitol |
| Gosogliptin | Exenatide |
| Gosogliptin | Liraglutide |
| Gosogliptin | Taspoglutide |
| Gosogliptin | Semaglutide |
| Gosogliptin | Albiglutide |
| Gosogliptin | Lixisenatide |
| Teneligliptin | selected from the group G3 |
| Teneligliptin | Metformin |
| Teneligliptin | Pioglitazone |
| Teneligliptin | Rosiglitazone |
| Teneligliptin | Glibenclamide |
| Teneligliptin | Glimepiride |
| Teneligliptin | Gliquidone |
| Teneligliptin | Nateglinide |
| Teneligliptin | Repaglinide |
| Teneligliptin | Acarbose |
| Teneligliptin | Voglibose |
| Teneligliptin | Miglitol |
| Teneligliptin | Exenatide |
| Teneligliptin | Liraglutide |
| Teneligliptin | Taspoglutide |
| Teneligliptin | Semaglutide |
| Teneligliptin | Albiglutide |
| Teneligliptin | Lixisenatide |
| Dutogliptin | selected from the group G3 |
| Dutogliptin | Metformin |
| Dutogliptin | Pioglitazone |
| Dutogliptin | Rosiglitazone |
| Dutogliptin | Glibenclamide |
| Dutogliptin | Glimepiride |
| Dutogliptin | Gliquidone |
| Dutogliptin | Nateglinide |
| Dutogliptin | Repaglinide |
| Dutogliptin | Acarbose |
| Dutogliptin | Voglibose |
| Dutogliptin | Miglitol |
| Dutogliptin | Exenatide |
| Dutogliptin | Liraglutide |
| Dutogliptin | Taspoglutide |
| Dutogliptin | Semaglutide |
| Dutogliptin | Albiglutide |
| Dutogliptin | Lixisenatide |

In a particular embodiment (embodiment E2) the pharmaceutical compositions, combinations, methods and uses according to this invention relate to combinations wherein the DPP-4 inhibitor is linagliptin. According to embodiment E2 the second antidiabetic agent is preferably selected according to the entries in the Table 2.

TABLE 2

| Embodiment | Second Antidiabetic Agent |
|---|---|
| E2.1 | selected from the group G3 |
| E2.2 | Metformin |
| E2.3 | Pioglitazone |
| E2.4 | Rosiglitazone |
| E2.5 | Glibenclamide |
| E2.6 | Glimepiride |
| E2.7 | Gliquidone |
| E2.8 | Nateglinide |
| E2.9 | Repaglinide |
| E2.10 | Acarbose |
| E2.11 | Voglibose |
| E2.12 | Miglitol |
| E2.13 | Exenatide |
| E2.14 | Liraglutide |
| E2.15 | Taspoglutide |
| E2.16 | Semaglutide |
| E2.17 | Albiglutide |
| E2.18 | Lixisenatide |

The combination of a DPP-4 inhibitor and a second and, optionally, a third antidiabetic agent according to this invention can be found to improve the glycemic control, in particular in patients as described hereinafter, compared with a monotherapy using either a DPP-4 inhibitor or the second or third antidiabetic agent alone, for example with a monotherapy of metformin, or with a dual therapy using the second and third antidiabetic agent. Further, the triple combination of a DPP-4 inhibitor and a second and a third antidiabetic agent according to this invention can be found to improve the glycemic control, in particular in patients as described hereinafter, compared with a combination therapy using a DPP-4 inhibitor and either the second or third antidiabetic agent, or using the second and the third antidiabetic agent. The improved glycemic control is determined as an increased lowering of blood glucose and an increased reduction of HbA1c. With monotherapy in a patient, in particular in patients as described hereinafter, the glycemic control may not be further improved significantly by an administration of the drug above a certain highest dose. In addition, a long term treatment using a highest dose may be unwanted in view of potential side effects. Therefore, a satisfying glycemic control may not be achievable in all patients via a monotherapy using either the DPP-4 inhibitor or the second or the third antidiabetic agent alone. In the case that monotherapy do not yield in full glycemic control, dual therapy may become necessary. Even with combination therapy using two agents selected from the DPP-4 inhibitors and second and third antidiabetic agents may not yield in a full glycemic control in all patients and/or over a long time. In the case that dual therapy do not yield in full glycemic control, triple therapy may become necessary. In such patients with inadequate glycemic control a progression of the diabetes mellitus may continue and complications associated with diabetes mellitus may occur, such as macrovascular complications. The pharmaceutical composition or combination as well as the methods according to the present invention allow a reduction of the HbA1c value to a desired target range, for example <7% and preferably <6.5%, for a higher number of patients and for a longer time of therapeutic treatment, e.g. in the case of dual or triple combination therapy compared with a monotherapy using one of or, respectively, a dual therapy using two of the combination partners.

In addition, the combination of a DPP-4 inhibitor and the second and, optionally, the third therapeutic agent according to this invention can be found to allow a reduction in the dose of either the DPP-4 inhibitor or the second or third antidiabetic agent or even of two or three of the active ingredients. A dose reduction is beneficial for patients which otherwise would potentially suffer from side effects in a therapy using a higher dose of one or more of the active ingredients, in particular with regard to side effect caused by the second and/or third antidiabetic agent. Therefore, the pharmaceutical combination as well as the methods according to the present invention, may show less side effects, thereby making the therapy more tolerable and improving the patients compliance with the treatment.

A DPP-4 inhibitor according to the present invention is able—via the increases in active GLP-1 levels—to reduce the glucagon secretion in a patient. This will therefore limit the hepatic glucose production. Furthermore, the elevated active GLP-1 levels produced by the DPP-4 inhibitor will have beneficial effects on beta-cell regeneration and neogenesis. All these features of DPP-4 inhibitors may render a pharmaceutical composition or combination or method of this invention quite useful and therapeutically relevant.

When this invention refers to patients requiring treatment or prevention, it relates primarily to treatment and prevention in humans, but the pharmaceutical composition may also be used accordingly in veterinary medicine in mammals. In the scope of this invention adult patients are preferably humans of the age of 18 years or older. Also in the scope of this invention, patients are adolescent humans, i.e. humans of age 10 to less than 18 years, preferably of age 13 to less than 18 years.

In an embodiment of this invention, a treatment or prophylaxis according to this invention is suitable in those patients in need of such treatment or prophylaxis who are diagnosed of one or more of the conditions selected from the group consisting of overweight and obesity, in particular class I obesity, class II obesity, class III obesity, visceral obesity and abdominal obesity. In addition a treatment or prophylaxis according to this invention is advantageously suitable in those patients in which a weight increase is contraindicated. Any weight increasing effect in the therapy, for example due to the administration of the second and/or third antidiabetic agent, may be attenuated or even avoided thereby.

In a further embodiment of this invention, the pharmaceutical composition or combination of this invention exhibits a very good efficacy with regard to glycemic control, in particular in view of a reduction of fasting plasma glucose, postprandial plasma glucose and/or glycosylated hemoglobin (HbA1c). By administering a pharmaceutical composition or combination according to this invention, a reduction of HbA1c equal to or greater than preferably 1.0%, more preferably equal to or greater than 2.0%, even more preferably equal to or greater than 3.0% can be achieved and the reduction is particularly in the range from 1.0% to 3.0%.

Furthermore, the method and/or use according to this invention is applicable in those patients who show one, two or more of the following conditions:
(a) a fasting blood glucose or serum glucose concentration greater than 110 mg/dL or greater than 100 mg/dL, in particular greater than 125 mg/dL;
(b) a postprandial plasma glucose equal to or greater than 140 mg/dL;
(c) an HbA1c value equal to or greater than 6.5%, in particular equal to or greater than 7.0%, especially equal to or greater than 7.5%, even more particularly equal to or greater than 8.0%.

The present invention also discloses the use of the pharmaceutical composition or combination for improving glycemic control in patients having type 2 diabetes or showing first signs of pre-diabetes. Thus, the invention also includes diabetes prevention. If therefore a pharmaceutical composition or combination of this invention is used to improve the glycemic control as soon as one of the above-mentioned signs of pre-diabetes is present, the onset of manifest type 2 diabetes mellitus can be delayed or prevented.

Furthermore, the pharmaceutical composition or combination of this invention is particularly suitable in the treatment of patients with insulin dependency, i.e. in patients who are treated or otherwise would be treated or need treatment with an insulin or a derivative of insulin or a substitute of insulin or a formulation comprising an insulin or a derivative or substitute thereof. These patients include patients with diabetes type 2 and patients with diabetes type 1.

Therefore, according to an embodiment of the present invention, there is provided a method for improving glycemic control and/or for reducing of fasting plasma glucose, of postprandial plasma glucose and/or of glycosylated hemoglobin HbA1c in a patient in need thereof who is diagnosed with impaired glucose tolerance (IGT), impaired fasting blood glucose (IFG) with insulin resistance, with metabolic syndrome and/or with type 2 or type 1 diabetes mellitus characterized in that a DPP-4 inhibitor and, optionally, a second and, optionally, a third antidiabetic agent as defined hereinbefore and hereinafter are administered, for example in combination, to the patient.

According to another embodiment of the present invention, there is provided a method for improving gycemic control in patients, in particular in adult patients, with type 2 diabetes mellitus as an adjunct to diet and exercise.

Unless otherwise noted, patients within the meaning of this invention may include drug naïve patients and/or drug pre-treated patients, e.g. patients treated with one or more conventional oral and/or non-oral antidiabetic drugs. Accordingly, unless otherwise noted, combination therapy within the meaning of this invention may include initial combination therapy, replacement and/or add-on combination therapy.

It can be found that by using a pharmaceutical composition or combination according to this invention, an improvement of the glycemic control can be achieved even in those patients who have insufficient glycemic control in particular despite treatment with the second or third antidiabetic agent or a combination of the second with the third antidiabetic agent, for example despite maximal tolerated dose of oral monotherapy with metformin or a combination of metformin with the third antidiabetic agent.

Thus, it can be found that by using a pharmaceutical composition or combination according to this invention, an improvement of the glycemic control can be achieved even in those patients who have insufficient glycemic control despite maximal tolerated dose of oral monotherapy with metformin, a thiazolidinedione (e.g. pioglitazone) or a sulfonylurea, or of oral combination therapy with metformin and a sulfonylurea, metformin with a thiazolidinedione (e.g. pioglitazone), or a thiazolidinedione (e.g. pioglitazone) with a sulfonylurea.

It can be further found that by using a combination according to this invention, an improvement of the glycemic control can be achieved even in those patients who have insufficient glycemic control in particular despite treatment with a DPP-4 inhibitor or a combination of a DPP-4 inhibitor with the second or third antidiabetic agent, for example despite maximal tolerated dose of oral monotherapy with a DPP-4 inhibitor or a dual combination of a DPP-4 inhibitor with the second or third antidiabetic agent.

A maximal tolerated dose with regard to metformin is for example 2000 mg per day, 1500 mg per day (for example in asian countries) or 850 mg three times a day or any equivalent thereof. A maximal tolerated dose with regard to sitagliptin is for example 100 mg once daily or any equivalent thereof.

Therefore, the method and/or use according to this invention is applicable in those patients who show one, two or more of the following conditions:
(a) insufficient glycemic control with diet and exercise alone;
(b) insufficient glycemic control despite oral monotherapy with metformin, in particular despite oral monotherapy at a maximal tolerated dose of metformin;
(c) insufficient glycemic control despite oral monotherapy with the second or third antidiabetic agent, in particular despite oral monotherapy at a maximal tolerated dose of the second or third antidiabetic agent;
(d) insufficient glycemic control despite combination therapy with two agents selected from the group of the second and third antidiabetic agent;
(e) insufficient glycemic control despite oral monotherapy with a thiazolidinedione, in particular despite oral monotherapy at a maximal tolerated dose of a thiazolidinedione (e.g. pioglitazone);
(f) insufficient glycemic control despite oral monotherapy with a sulfonylurea, in particular despite oral monotherapy at a maximal tolerated dose of a sulfonylurea;
(g) insufficient glycemic control despite combination therapy with two agents selected from the group consisting of metformin, a thiazolidinedione (e.g. pioglitazone) and a sulfonylurea, for example despite combination therapy with a dual combination selected from metformin/pioglitazone, metformin/sulphonylurea, and sulphonylurea/pioglitazone.

The method and/or use according to this invention is further applicable in those patients who show one or more of the following conditions:
(h) insufficient glycemic control despite therapy on insulin (e.g. with or without further conventional oral antidiabetic drug);
(i) insufficient glycemic control despite combination therapy with insulin and the second and/or third antidiabetic agent, in particular despite combination therapy with insulin and maximal tolerated dose of metformin, a thiazolidinedione (e.g. pioglitazone) or a sulfonylurea, for example despite combination therapy with a dual combination selected from metformin/insulin, sulphonylurea/insulin, and pioglitazone/insulin.

The dual or triple combination method and/or use according to this invention is further applicable in those patients who show the following conditions (j) or (k), respectively:
(j) insufficient glycemic control despite oral monotherapy with the DPP-4 inhibitor, in particular despite oral monotherapy at a maximal tolerated dose of the DPP-4 inhibitor;
(k) insufficient glycemic control despite oral combination therapy with the DPP-4 inhibitor and the second or third antidiabetic agent, in particular despite oral dual therapy at a maximal tolerated dose of at least one of the combination partners.

In an embodiment of this invention, a pharmaceutical composition or combination is suitable in the treatment of patients who are diagnosed having one or more of the following conditions
insulin resistance,
hyperinsulinemia,
pre-diabetes,
type 2 diabetes mellitus, particular having a late stage type 2 diabetes mellitus,
type 1 diabetes mellitus.

Furthermore, a pharmaceutical composition or combination according to this invention is particularly suitable in the treatment of patients who are diagnosed having one or more of the following conditions
(a) obesity (including class I, II and/or III obesity), visceral obesity and/or abdominal obesity,
(b) triglyceride blood level >150 mg/dL,
(c) HDL-cholesterol blood level <40 mg/dL in female patients and <50 mg/dL in male patients,
(d) a systolic blood pressure >130 mm Hg and a diastolic blood pressure >85 mm Hg,
(e) a fasting blood glucose level >110 mg/dL or >100 mg/dL.

It is assumed that patients diagnosed with impaired glucose tolerance (IGT), impaired fasting blood glucose (IFG), with insulin resistance and/or with metabolic syndrome suffer from an increased risk of developing a cardiovascular disease, such as for example myocardial infarction, coronary heart disease, heart insufficiency, thromboembolic events. A glycemic control according to this invention may result in a reduction of the cardiovascular risks.

Furthermore, the pharmaceutical composition and the methods according to this invention are particularly suitable in the treatment of patients after organ transplantation, in particular those patients who are diagnosed having one or more of the following conditions
(a) a higher age, in particular above 50 years,
(b) male gender;
(c) overweight, obesity (including class I, II and/or III obesity), visceral obesity and/or abdominal obesity,
(d) pre-transplant diabetes,
(e) immunosuppression therapy.

A pharmaceutical composition or combination according to this invention, in particular due to the DPP-4 inhibitor therein, exhibits a good safety profile. Therefore, a treatment or prophylaxis according to this invention is possible in those patients for which the mono-therapy with another antidiabetic drug, such as for example metformin, is contraindicated and/or who have an intolerance against such drugs at therapeutic doses. In particular, a treatment or prophylaxis according to this invention may be advantageously possible in those patients showing or having an increased risk for one or more of the following disorders: renal insufficiency or diseases, cardiac diseases, cardiac failure, hepatic diseases, pulmonal diseases, catabolytic states and/or danger of lactate acidosis, or female patients being pregnant or during lactation.

Furthermore, it can be found that the administration of a pharmaceutical composition or combination according to this invention results in no risk or in a low risk of hypoglycemia. Therefore, a treatment or prophylaxis according to this invention is also advantageously possible in those patients showing or having an increased risk for hypoglycemia.

A pharmaceutical composition or combination according to this invention is particularly suitable in the long term treatment or prophylaxis of the diseases and/or conditions as described hereinbefore and hereinafter, in particular in the long term glycemic control in patients with type 2 diabetes mellitus.

The term “long term” as used hereinbefore and hereinafter indicates a treatment of or administration in a patient within a period of time longer than 12 weeks, preferably longer than 25 weeks, even more preferably longer than 1 year.

Therefore, a particular embodiment of the present invention provides a method for therapy, preferably oral therapy, for improvement, especially long term improvement, of glycemic control in patients with type 2 diabetes mellitus, especially in patients with late stage type 2 diabetes mellitus, in particular in patients additionally diagnosed of overweight, obesity (including class I, class II and/or class III obesity), visceral obesity and/or abdominal obesity.

The effects mentioned above are observed both, when the DPP-4 inhibitor and the second and, optionally, third antidiabetic agent are administered together, for example simultaneously in one single or two or three separate formulations, and/or when they are administered in alternation, for example successively in two or three separate formulations.

Within this invention it is to be understood that combinations or combined uses envisage the separate, sequential, simultaneous, concurrent, chronologically staggered or alternating administration of the components. It will be appreciated that the DPP-4 inhibitor and the other active substance(s) can be administered in a single dosage form or each in separate dosage forms.

In this context, “combination” or “combined” within the meaning of this invention also includes, without being limited, fixed and non-fixed forms and uses.

It will be appreciated that the amount of the pharmaceutical composition according to this invention to be administered to the patient and required for use in treatment or prophylaxis according to the present invention will vary with the route of administration, the nature and severity of the condition for which treatment or prophylaxis is required, the age, weight and condition of the patient, concomitant medication and will be ultimately at the discretion of the attendant physician. In general, however, the DPP-4 inhibitor and, optionally, the second and/or third antidiabetic agent according to this invention are included in the pharmaceutical composition, combination or dosage form in an amount sufficient that by their administration the glycemic control in the patient to be treated is improved.

In the following preferred ranges of the amount of the DPP-4 inhibitor, the second and/or third antidiabetic agent to be employed in the pharmaceutical composition and the methods and uses according to this invention are described. These ranges refer to the amounts to be administered per day with respect to an adult patient, in particular to a human being, for example of approximately 70 kg body weight, and can be adapted accordingly with regard to an administration 2, 3, 4 or more times daily and with regard to other routes of administration and with regard to the age of the patient. The ranges of the dosage and amounts are calculated for the individual active moiety. Advantageously, the combination therapy of the present invention utilizes lower dosages of the individual DPP-4 inhibitor and/or of the individual second and/or third antidiabetic agent used in monotherapy or used in conventional therapeutics, thus avoiding possible toxicity and adverse side effects incurred when those agents are used as monotherapies.

Within the scope of the present invention, the pharmaceutical composition or combination is preferably administered orally. Other forms of administration are possible and described hereinafter. Preferably the one or more dosage forms comprising the DPP-4 inhibitor and/or the second and/or the third antidiabetic agent is oral or usually well known.

In general, the amount of the DPP-4 inhibitor in the combinations, combination methods or combined uses of this invention is preferably in the range from 1/5 to 1/1 of the amount usually recommended for a monotherapy using said DPP-4 inhibitor.

A preferred dosage range of linagliptin when administered orally is 0.5 mg to 10 mg per day, preferably 2.5 mg to 10 mg, most preferably 1 mg to 5 mg per day. The preferred range of amounts in the pharmaceutical composition is 0.5 to 10 mg, in particular 1 to 5 mg. Examples of particular dosage strengths are 1, 2.5, 5 or 10 mg. The application of the active ingredient may occur up to three times a day, preferably one or two times a day. Suitable formulations for linagliptin may be those formulations disclosed in the application WO 2007/128724, the disclosure of which is incorporated herein in its entirety.

Typical dosage strengths of the dual combination of linagliptin/metformin are 2.5/500 mg, 2.5/850 mg and 2.5/1000 mg, which may be administered 1-3 times a day, particularly twice a day.

A preferred dosage range of sitagliptin when administered orally is from 10 to 200 mg, in particular 25 to 150 mg per day. A recommended dose of sitagliptin is 100 mg calculated for the active moiety (free base anhydrate) once daily or 50 mg twice daily. The preferred range of amounts in the pharmaceutical composition is 10 to 150 mg, in particular 25 to 100 mg. Examples are 25, 50, 75 or 100 mg. The application of the active ingredient may occur up to three times a day, preferably one or two times a day. Equivalent amounts of salts of sitagliptin, in particular of the phosphate monohydrate can be calculated accordingly. Adjusted dosages of sitagliptin, for example 25 and 50 mg, are preferably used for patients with renal failure. Typical dosage strengths of the dual combination of sitagliptin/metformin are 50/500 mg and 50/1000 mg.

A preferred dosage range of vildagliptin when administered orally is from 10 to 150 mg daily, in particular from 25 to 150 mg, 25 and 100 mg or 25 and 50 mg or 50 and 100 mg daily. For example the daily administration of vildagliptin is 50 or 100 mg. The preferred range of amounts in the pharmaceutical composition is 10 to 150 mg, in particular 25 to 100 mg. Examples are 25, 50, 75 or 100 mg. The application of the active ingredient may occur up to three times a day, preferably one or two times a day. Typical dosage strengths of the dual combination of vildagliptin/metformin are 50/850 mg and 50/1000 mg.

A preferred dosage range of alogliptin when administered orally is from 5 to 250 mg daily, in particular from 10 to 150 mg daily. The preferred range of amounts in the pharmaceutical composition is 5 to 150 mg, in particular 10 to 100 mg. Examples are 10, 12.5, 20, 25, 50, 75 and 100 mg. The application of the active ingredient may occur up to three times a day, preferably one or two times a day.

A preferred dosage range of saxagliptin when administered orally is from 2.5 to 100 mg daily, in particular from 2.5 to 50 mg daily. The preferred range of amounts in the pharmaceutical composition is from 2.5 to 100 mg, in particular from 2.5 and 50 mg. Examples are 2.5, 5, 10, 15, 20, 30, 40, 50 and 100 mg. The application of the active ingredient may occur up to three times a day, preferably one or two times a day. Typical dosage strengths of the dual combination of saxagliptin/metformin are 2.5/500 mg and 2.5/1000 mg.

A preferred dosage range of dutogliptin when administered orally is from 50 to 400 mg daily, in particular from 100 to 400 mg daily. The preferred range of amounts in the pharmaceutical composition is from 50 to 400 mg. Examples are 50, 100, 200, 300 and 400 mg. The application of the active ingredient may occur up to three times a day, preferably one or two times a day.

A special embodiment of the DPP-4 inhibitors of this invention refers to those orally administered DPP-4 inhibitors which are therapeutically efficacious at low dose levels, e.g. at dose levels <100 mg or <70 mg per patient per day, preferably <50 mg, more preferably <30 mg or <20 mg, even more preferably from 1 mg to 10 mg (if required, divided into 1 to 4 single doses, particularly 1 or 2 single doses, which may be of the same size), particularly from 1 mg to 5 mg (more particularly 5 mg), per patient per day, preferentially, administered orally once-daily, more preferentially, at any time of day, administered with or without food. Thus, for example, the daily oral amount 5 mg BI 1356 can be given in a once daily dosing regimen (i.e. 5 mg BI 1356 once daily) or in a twice daily dosing regimen (i.e. 2.5 mg BI 1356 twice daily), at any time of day, with or without food.

In general, the amount of the second and/or third antidiabetic agent in the combinations, combination methods and/or combined uses of this invention is preferably in the range from 1/5 to 1/1 of the amount usually recommended for a monotherapy using said antidiabetic agent. Using lower dosages of the individual second and/or third antidiabetic agent compared with monotherapy could avoid or minimize possible toxicity and adverse side effects incurred when those agents are used as monotherapies.

A preferred dosage range of metformin when administered orally is 250 to 3000 mg, in particular 500 to 2000 mg per day. The preferred range of amounts in the pharmaceutical composition is 250 to 1000, in particular 500 to 1000 mg or 250 to 850 mg respectively. Examples are 500, 750, 850 or 1000 mg. Preferably the administration of said amounts is once, twice or three times daily. For example the amounts of 500, 750 and 850 mg preferably require once-daily, twice-daily or three-times daily dosing and the amount of 1000 mg preferably requires once-daily or twice-daily dosing. Certain controlled or sustained release formulations allow a once-daily dosing. Metformin can be administered for example in the form as marketed under the trademarks GLUCOPHAGE™, GLUCOPHAGE-D™ or GLUCOPHAGE-XR™

A preferred dosage range of pioglitazone when administered orally is 5 to 50 mg per day. The preferred range of amounts in the pharmaceutical composition is 5 to 50 mg, 10 to 45 mg and 15 to 45 mg respectively. Examples are 15, 30 or 45 mg. Preferably the administration of said amounts is once or twice daily, in particular once daily. Pioglitazone can be administered in the form as it is marketed for example under the trademark ACTOS™.

A preferred dosage range of rosiglitazone when administered orally is 1 mg to 10 mg per day. The preferred range of amounts in the pharmaceutical composition is 1 to 10 mg, 2 to 8 mg, 4 to 8 mg and 1 to 4 mg. Examples are 1, 2, 4 or 8 mg. Preferably the administration of said amounts is once or twice daily. Preferably the dose should not exceed 8 mg daily. Rosiglitazone can be administered in the form as it is marketed for example under the trademark AVANDIA™.

A preferred dosage range of a thiazolidindione (other than pioglitazone or rosiglitazone as described above) when administered orally is 2 to 100 mg per day. The preferred range of amounts in the pharmaceutical composition for an administration once, twice or three times daily is 2 to 100, 1 to 50 and 1 to 33 mg respectively.

A preferred dosage range of glibenclamide when administered orally is 0.5 to 15 mg, in particular 1 to 10 mg per day. The preferred range of amounts in the pharmaceutical composition is 0.5 to 5 mg, in particular 1 to 4 mg. Examples are 1.0, 1.75 and 3.5 mg. Preferably the administration of said amounts is once, twice or three-times daily. Glibenclamide can be administered in the form as it is marketed for example under the trademark EUGLUCON™.

A preferred dosage range of glimepiride when administered orally is 0.5 to 10 mg, in particular 1 to 6 mg per day. The preferred range of amounts in the pharmaceutical composition is 0.5 to 10 mg, in particular 1 to 6 mg. Examples are 1, 2, 3, 4, and 6 mg. Preferably the administration of said amounts is once, twice or three-times daily, preferably once daily. Glimepiride can be administered in the form as it is marketed for example under the trademark AMARYL™.

A preferred dosage range of gliquidone when administered orally is 5 to 150 mg, in particular 15 to 120 mg per day. The preferred range of amounts in the pharmaceutical composition is 5 to 120 mg, in particular 5 to 30 mg. Examples are 10, 20, 30 mg. Preferably the administration of said amounts is once, twice, three-times or four-times daily. Gliquidone can be administered in the form as it is marketed for example under the trademark GLURENORM™.

A preferred dosage range of glibornuride when administered orally is 5 to 75 mg per day. The preferred range of amounts in the pharmaceutical composition is 5 to 75 mg, in particular 10 to 50 mg. Preferably the administration of said amounts is once, twice or three-times daily.

A preferred dosage range of gliclazide when administered orally is 20 to 300 mg, in particular 40 to 240 mg per day. The preferred range of amounts in the pharmaceutical composition is 20 to 240 mg, in particular 20 to 80 mg. Examples are 20, 30, 40 and 50 mg. Preferably the administration of said amounts is once, twice or three-times daily.

A preferred dosage range of glisoxepide when administered orally is 1 to 20 mg, in particular 1 to 16 mg per day. The preferred range of amounts in the pharmaceutical composition is 1 to 8 mg, in particular 1 to 4 mg. Preferably the administration of said amounts is once, twice, three-times or four-times daily.

A preferred dosage range of tolbutamide when administered orally is 100 to 3000 mg, preferably 500 to 2000 mg per day. The preferred range of amounts in the pharmaceutical composition is 100 to 1000 mg. Preferably the administration of said amounts is once or twice daily.

A preferred dosage range of glipizide when administered orally is 1 to 50 mg, in particular 2.5 to 40 mg per day. The preferred range of amounts in the pharmaceutical composition for an administration once, twice or three times daily is 1 to 50, 0.5 to 25 and 0.3 to 17 mg respectively.

A preferred dosage range of nateglinide when administered orally is 30 to 500 mg, in particular 60 to 360 mg per day. The preferred range of amounts in the pharmaceutical composition is 30 to 120 mg. Examples are 30, 60 and 120 mg. Preferably the administration of said amounts is once, twice or three-times daily. Nateglinide can be administered in the form as it is marketed for example under the trademark STARLIX™.

A preferred dosage range of repaglinide when administered orally is 0.1 to 16 mg, in particular 0.5 to 6 mg per day.

The preferred range of amounts in the pharmaceutical composition is 0.5 to 4 mg. Examples are 0.5, 1, 2 or 4 mg. Preferably the administration of said amounts is once, twice, three-times or four-times daily. Repaglinide can be administered in the form as it is marketed for example under the trademark NOVONORM™.

A preferred dosage range of acarbose when administered orally is 50 to 1000 mg, in particular 50 to 600 mg per day. The preferred range of amounts in the pharmaceutical composition is 50 to 150 mg. Examples are 50 and 100 mg. Preferably the administration of said amounts is once, twice, three-times or four-times daily. Acarbose can be administered in the form as it is marketed for example under the trademark Glucobay™.

A preferred dosage range of voglibose when administered orally is 100 to 1000 mg, in particular 200 to 600 mg per day. The preferred range of amounts in the pharmaceutical composition is 50 to 300 mg. Examples are 50, 100, 150, 200 and 300 mg. Preferably the administration of said amounts is once, twice, three-times or four-times daily. Voglibose can be administered in the form as it is marketed for example under the trademark Basen™ or Voglisan™.

A preferred dosage range of miglitol when administered orally is 25 to 500 mg, in particular 25 to 300 mg per day. The preferred range of amounts in the pharmaceutical composition is 25 to 100 mg. Examples are 25, 50 and 100 mg. Preferably the administration of said amounts is once, twice, three-times or four-times daily. Miglitol can be administered in the form as it is marketed for example under the trademark Glyset™.

A preferred dosage range of GLP-1 analogues, in particular of exenatide is 5 to 30 μg, in particular 5 to 20 μg per day. The preferred range of amounts in the pharmaceutical composition is 5 to 10 μg. Examples are 5 and 10 μg. Preferably the administration of said amounts is once, twice, three-times or four-times daily by subcutaneous injection. Exenatide can be administered in the form as it is marketed for example under the trademark Byetta™. A long acting formulation, preferably for a once weekly subcutaneous injection, comprises an amount from 0.1 to 3.0 mg, preferably 0.5 to 2.0 mg exenatide. Examples are 0.8 mg and 2.0 mg. An example of a long acting formulation of exenatide is Byetta LAR™.

A preferred dosage range of liraglutide is 0.5 to 3 mg, in particular 0.5 to 2 mg per day. The preferred range of amounts in the pharmaceutical composition is 0.5 to 2 mg. Examples are 0.6, 1.2 and 1.8 mg. Preferably the administration of said amounts is once or twice daily by subcutaneous injection.

The amount of the DPP-4 inhibitor and the second and/or third therapeutic agent in the pharmaceutical composition and in the methods and uses of this invention correspond to the respective dosage ranges as provided hereinbefore. For example, preferred dosage ranges in a pharmaceutical composition, combination, method and use according to this invention are an amount of 0.5 to 10 mg (in particular 1 to 5 mg, especially 2.5 mg or 5 mg) of linagliptin and/or an amount of 250 to 1000 mg (especially 500 mg, 850 mg or 1000 mg) of metformin. An oral administration once or twice daily is preferred.

In the combination methods and combined uses according to the present invention the DPP-4 inhibitor and the second and/or third therapeutic agent are administered in combination including, without being limited, the active ingredients are administered at the same time, i.e. simultaneously, or essentially at the same time, or the active ingredients are administered in alternation, i.e. that at first one or two active ingredients are administered and after a period of time the other two or one active ingredients are administered, i.e. at least two of the three active ingredients are administered sequentially. The period of time may be in the range from 30 min to 12 hours. The administration which is in combination or in alternation may be once, twice, three times or four times daily, preferably once or twice daily.

With regard to combined administration of the DPP-4 inhibitor and the second and/or third antidiabetic agent, all three active ingredients may be present in one single dosage form, for example in one tablet or capsule, or one or two of the active ingredients may be present in a separate dosage form, for example in two different or identical dosage forms.

With regard to their administration in alternation, one or two of the active ingredients are present in a separate dosage form, for example in two different or identical dosage forms.

Therefore, a pharmaceutical combination of this invention may be present as single dosage forms which comprise the DPP-4 inhibitor and the second and, optionally, the third antidiabetic agent. Alternatively a pharmaceutical combination of this invention may be present as two separate dosage forms wherein one dosage form comprises the DPP-4 inhibitor and the other dosage form comprises the second plus, optionally, the third antidiabetic agent, or, in case of a triple combination, one dosage form comprises the DPP-4 inhibitor plus either the second or the third antidiabetic agent and the other dosage form comprises the third or the second antidiabetic agent, respectively. Alternatively, in case of a triple combination, a pharmaceutical combination of this invention may be present as three separate dosage forms wherein one dosage form comprises the DPP-4 inhibitor and a second dosage form comprises the second antidiabetic agent and the third dosage form comprises the third antidiabetic agent. Alternatively, in case of a dual combination, a pharmaceutical combination of this invention may be present as two separate dosage forms wherein one dosage form comprises the DPP-4 inhibitor and the second dosage form comprises the second antidiabetic agent.

The case may arise in which an active ingredient has to be administered more often, for example twice per day, than the other active ingredient(s), which for example needs administration once daily. Therefore “administration in combination” also includes an administration scheme in which first all active ingredients are administered in combination and after a period of time an active ingredient is administered again or vice versa.

Therefore, the present invention also includes pharmaceutical combinations which are present in separate dosage forms wherein one dosage form comprises the DPP-4 inhibitor and the second and, optionally, the third, therapeutic agent and the other dosage form comprises the second and/or the third therapeutic agent only.

Thus, the present invention also includes pharmaceutical compositions or combinations for separate, sequential, simultaneous, concurrent, alternate or chronologically staggered use of the active ingredients.

A pharmaceutical composition which is present as a separate or multiple dosage form, preferably as a kit of parts, is useful in combination therapy to flexibly suit the individual therapeutic needs of the patient.

According to a first embodiment a kit of parts comprises
(a) a first containment containing a dosage form comprising the DPP-4 inhibitor and at least one pharmaceutically acceptable carrier, and
(b) a second containment containing a dosage form comprising the second antidiabetic agent and at least one pharmaceutically acceptable carrier, and, optionally,
(c) a third containment containing a dosage form comprising the third antidiabetic agent and at least one pharmaceutically acceptable carrier.

According to a second embodiment a kit of parts comprises
(a) a first containment containing a dosage form comprising the DPP-4 inhibitor and the second or third antidiabetic agent and at least one pharmaceutically acceptable carrier, and
(b) a second containment containing a dosage form comprising the third or second antidiabetic agent, respectively, and at least one pharmaceutically acceptable carrier.

According to a third embodiment a kit of parts comprises
(a) a first containment containing a dosage form comprising the DPP-4 inhibitor and at least one pharmaceutically acceptable carrier, and
(b) a second containment containing a dosage form comprising the second and third antidiabetic agent and at least one pharmaceutically acceptable carrier.

A further aspect of the present invention is a manufacture comprising the pharmaceutical combination being present as separate dosage forms according to the present invention and a label or package insert comprising instructions that the separate dosage forms are to be administered in combination.

According to a first embodiment a manufacture comprises (a) a pharmaceutical composition comprising a DPP-4 inhibitor according to the present invention and (b) a label or package insert which comprises instructions that the medicament may or is to be administered, for example in combination, with a medicament comprising a second antidiabetic agent according to the present invention or with a fixed or free combination (e.g. a medicament) comprising a second antidiabetic agent and a third antidiabetic agent according to the present invention.

According to a second embodiment a manufacture comprises (a) a second antidiabetic agent according to the present invention and (b) a label or package insert which comprises instructions that the medicament may or is to be administered, for example in combination, with a medicament comprising a DPP-4 inhibitor according to the present invention or with a a fixed or free-combination (e.g. a medicament) comprising a DPP-4 inhibitor and a third antidiabetic agent according to the present invention.

According to a third embodiment a manufacture comprises (a) a pharmaceutical composition comprising a DPP-4 inhibitor and a second antidiabetic agent according to the present invention and (b) a label or package insert which comprises instructions that the medicament may or is to be administered, for example in combination, with a medicament comprising a third antidiabetic agent according to the present invention.

The desired dose of the pharmaceutical composition according to this invention may conveniently be presented in a once daily or as divided dose administered at appropriate intervals, for example as two, three or more doses per day.

The pharmaceutical composition may be formulated for oral, rectal, nasal, topical (including buccal and sublingual), transdermal, vaginal or parenteral (including intramuscular, subcutaneous and intravenous) administration in liquid or solid form or in a form suitable for administration by inhalation or insufflation. Oral administration is preferred. The formulations may, where appropriate, be conveniently presented in discrete dosage units and may be prepared by any of the methods well known in the art of pharmacy. All methods include the step of bringing into association the active ingredient with one or more pharmaceutically acceptable carriers, like liquid carriers or finely divided solid carriers or both, and then, if necessary, shaping the product into the desired formulation.

The pharmaceutical composition may be formulated in the form of tablets, granules, fine granules, powders, capsules, caplets, soft capsules, pills, oral solutions, syrups, dry syrups, chewable tablets, troches, effervescent tablets, drops, suspension, fast dissolving tablets, oral fast-dispersing tablets, etc.

The pharmaceutical composition and the dosage forms preferably comprises one or more pharmaceutical acceptable carriers. Preferred carriers must be “acceptable” in the sense of being compatible with the other ingredients of the formulation and not deleterious to the recipient thereof. Examples of pharmaceutically acceptable carriers are known to the one skilled in the art.

Pharmaceutical compositions suitable for oral administration may conveniently be presented as discrete units such as capsules, including soft gelatin capsules, cachets or tablets each containing a predetermined amount of the active ingredient; as a powder or granules; as a solution, a suspension or as an emulsion, for example as syrups, elixirs or self-emulsifying delivery systems (SEDDS). The active ingredients may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The pharmaceutical composition according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilisation from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the active compound(s) with the softened or melted carrier(s) followed by chilling and shaping in moulds.

For pharmaceutical application in warm-blooded vertebrates, particularly humans, the compounds of this invention are usually used in dosages from 0.001 to 100 mg/kg body weight, preferably at 0.1-15 mg/kg, in each case 1 to 4 times a day. For this purpose, the compounds, optionally combined with other active substances, may be incorporated together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof into conventional galenic preparations such as plain or coated tablets, capsules, powders, suspensions or suppositories.

The pharmaceutical compositions according to this invention comprising the DPP-4 inhibitors as defined herein are thus prepared by the skilled person using pharmaceutically acceptable formulation excipients as described in the art. Examples of such excipients include, without being restricted to diluents, binders, carriers, fillers, lubricants, flow promoters, crystallisation retardants, disintegrants, solubilizers, colorants, pH regulators, surfactants and emulsifiers.

Examples of suitable diluents for compounds according to embodiment A include cellulose powder, calcium hydrogen phosphate, erythritol, low substituted hydroxypropyl cellulose, mannitol, pregelatinized starch or xylitol. Among those diluents mannitol, low substituted hydroxypropyl cellulose and pregelatinized starch are to be emphasized.

Examples of suitable lubricants for compounds according to embodiment A include talc, polyethyleneglycol, calcium behenate, calcium stearate, hydrogenated castor oil or magnesium stearate. Among those lubricants magnesium stearate is to be emphasized.

Examples of suitable binders for compounds according to embodiment A include copovidone (copolymerisates of vinylpyrrolidon with other vinylderivates), hydroxypropyl methylcellulose (HPMC), hydroxypropylcellulose (HPC), polyvinylpyrrolidon (povidone), pregelatinized starch, or low-substituted hydroxypropylcellulose (L-HPC). Among those binders copovidone and pregelatinized starch are to be emphasized.

Examples of suitable disintegrants for compounds according to embodiment A include corn starch or crospovidone. Among those disintegrants corn starch is to be emphasized.

Suitable methods of preparing pharmaceutical formulations of the DPP-4 inhibitors according to embodiment A of the invention are
direct tabletting of the active substance in powder mixtures with suitable tabletting excipients;
granulation with suitable excipients and subsequent mixing with suitable excipients and subsequent tabletting as well as film coating; or
packing of powder mixtures or granules into capsules.

Suitable granulation methods are
wet granulation in the intensive mixer followed by fluidised bed drying;
one-pot granulation;
fluidised bed granulation; or
dry granulation (e.g. by roller compaction) with suitable excipients and subsequent tabletting or packing into capsules.

An exemplary composition of a DPP-4 inhibitor according to embodiment A of the invention comprises the first diluent mannitol, pregelatinized starch as a second diluent with additional binder properties, the binder copovidone, the disintegrant corn starch, and magnesium stearate as lubricant; wherein copovidone and/or corn starch may be optional.

For details on dosage forms, formulations and administration of DPP-4 inhibitors of this invention, reference is made to scientific literature and/or published patent documents, particularly to those cited herein.

The pharmaceutical compositions (or formulations) may be packaged in a variety of ways. Generally, an article for distribution includes a container that contains the pharmaceutical composition in an appropriate form. Tablets are typically packed in an appropriate primary package for easy handling, distribution and storage and for assurance of proper stability of the composition at prolonged contact with the environment during storage. Primary containers for tablets may be bottles or blister packs.

A suitable bottle, e.g. for a pharmaceutical composition or combination comprising a DPP-4 inhibitor according to embodiment A of the invention, may be made from glass or polymer (preferably polypropylene (PP) or high density polyethylene (HD-PE)) and sealed with a screw cap. The screw cap may be provided with a child resistant safety closure (e.g. press-and-twist closure) for preventing or hampering access to the contents by children. If required (e.g. in regions with high humidity), by the additional use of a desiccant (such as e.g. bentonite clay, molecular sieves, or, preferably, silica gel) the shelf life of the packaged composition can be prolonged.

A suitable blister pack, e.g. for a pharmaceutical composition or combination comprising a DPP-4 inhibitor according to embodiment A of the invention, comprises or is formed of a top foil (which is breachable by the tablets) and a bottom part (which contains pockets for the tablets). The top foil may contain a metalic foil, particularly an aluminium or aluminium alloy foil (e.g. having a thickness of 20 μm to 45 μm, preferably 20 μm to 25 μm) that is coated with a heat-sealing polymer layer on its inner side (sealing side). The bottom part may contain a multi-layer polymer foil (such as e.g. poly(vinyl choride) (PVC) coated with poly(vinylidene choride) (PVDC); or a PVC foil laminated with poly(chlorotriflouroethylene) (PCTFE)) or a multi-layer polymer-metal-polymer foil (such as e.g. a cold-formable laminated PVC/aluminium/polyamide composition).

The article may further comprise a label or package insert, which refer to instructions customarily included in commercial packages of therapeutic products, that may contain information about the indications, usage, dosage, administration, contraindications and/or warnings concerning the use of such therapeutic products. In one embodiment, the label or package inserts indicates that the composition can be used for any of the purposes described herein.

The pharmaceutical compositions and methods according to this invention show advantageous effects in the treatment and prevention of those diseases and conditions as described hereinbefore. The dual combinations show advantageous effects compared with monotherapy with an active ingredient. The triple combinations show advantageous effects compared with dual therapy with one or two of the three active ingredients. Advantageous effects may be seen for example with respect to efficacy, dosage strength, dosage frequency, pharmacodynamic properties, pharmacokinetic properties, fewer adverse effects, convenience, compliance, etc.

With respect to linagliptin, the methods of synthesis are known to the skilled person and as described in the literature, in particular as described in WO 2002/068420, WO 2004/018468, or WO 2006/048427, the disclosures of which are incorporated herein. Polymorphous crystal modifications and formulations of particular DPP-4 inhibitors are disclosed in WO 2007/128721 and WO 2007/128724, respectively, the disclosures of which are incorporated herein in their entireties. Formulations of particular DPP-4 inhibitors with metformin or other combination partners are described in WO 2009/121945, the disclosure of which is incorporated herein in its entirety.

The methods of synthesis for the further DPP-4 inhibitors are described in the scientific literature and/or in published patent documents, particularly in those cited hereinbefore.

The active ingredients, in particular the DPP-4 inhibitor and/or the second and/or the third antidiabetic agent, may be present in the form of a pharmaceutically acceptable salt.

Pharmaceutically acceptable salts include, without being restricted thereto, such as salts of inorganic acid like hydrochloric acid, sulfuric acid and phosphoric acid; salts of organic carboxylic acid like oxalic acid, acetic acid, citric acid, malic acid, benzoic acid, maleic acid, fumaric acid, tartaric acid, succinic acid and glutamic acid and salts of organic sulfonic acid like methanesulfonic acid and p-toluenesulfonic acid. The salts can be formed by combining the compound and an acid in the appropriate amount and ratio in a solvent and decomposer. They can be also obtained by the cation or anion exchange from the form of other salts.

The active ingredients or a pharmaceutically acceptable salt thereof may be present in the form of a solvate such as a hydrate or alcohol adduct.

Any of the above mentioned active substances, combinations and methods within the scope of the invention may be tested by animal models known in the art. In the following, in vivo experiments are described which are suitable to evaluate pharmacologically relevant properties of DPP-4 inhibitors, pharmaceutical compositions, combinations and methods according to this invention:

DPP-4 inhibitors, pharmaceutical compositions, combinations and methods according to this invention can be tested in genetically hyperinsulinemic or diabetic animals like db/db mice, ob/ob mice, Zucker Fatty (fa/fa) rats or Zucker Diabetic Fatty (ZDF) rats. In addition, they can be tested in animals with experimentally induced diabetes like HanWistar or Sprague Dawley rats pretreated with streptozotocin.

The effect on glycemic control of the combinations according to this invention can be tested after single dosing of the DPP-4 inhibitor and the second and, optionally, the third antidiabetic agent alone and in combination in an oral glucose tolerance test in the animal models described hereinbefore. The time course of blood glucose is followed after an oral glucose challenge in overnight fasted animals. The combinations according to the present invention may significantly improve glucose excursion compared to each monotherapy or, respectively, dual-combination therapy using a combination of two of the three active ingredients as measured by reduction of peak glucose concentrations or reduction of glucose AUC. In addition, after multiple dosing of the DPP-4 inhibitor and the second and, optionally, the third therapeutic agent alone and in combination in the animal models described hereinbefore, the effect on glycemic control can be determined by measuring the HbA1c value in blood. The combinations according to this invention may significantly reduce HbA1c compared to each monotherapy or, respectively, compared to a dual-combination therapy, i.e. using a combination of two of the three active ingredients.

The possible dose reduction of one or more of the DPP-4 inhibitor, the second and the third antidiabetic agent can be tested by the effect on glycemic control of lower doses of the combinations and monotherapies or dual-combination therapies in the animal models described hereinbefore. The combinations according to this invention at the lower doses may significantly improve glycemic control compared to placebo treatment whereas the monotherapies or, respectively, dual-combination therapies at lower doses do not.

An increase in active GLP-1 levels by treatment according to this invention after single or multiple dosing can be determined by measuring those levels in the plasma of animal models described hereinbefore in either the fasting or postprandial state. Likewise, a reduction in glucagon levels in plasma can be measured under the same conditions.

A superior effect of a DPP-4 inhibitor alone or in combination with a second and, optionally, a third antidiabetic agent according to the present invention on beta-cell regeneration and neogenesis can be determined after multiple dosing in the animal models described hereinbefore by measuring the increase in pancreatic insulin content, or by measuring increased beta-cell mass by morphometric analysis after immunhistochemical staining of pancreatic sections, or by measuring increased glucose-stimulated insulin secretion in isolated pancreatic islets.

As different metabolic functional disorders often occur simultaneously, it is quite often indicated to combine a number of different active principles with one another. Thus, depending on the functional disorders diagnosed, improved treatment outcomes may be obtained if a DPP-4 inhibitor is combined with active substances customary for the respective disorders, such as e.g. one or more active substances selected from among the other antidiabetic substances, especially active substances that lower the blood sugar level or the lipid level in the blood, raise the HDL level in the blood, lower blood pressure or are indicated in the treatment of atherosclerosis or obesity.

The DPP-4 inhibitors mentioned above—besides their use in mono-therapy—may also be used in conjunction with other active substances, by means of which improved treatment results can be obtained. Such a combined treatment may be given as a free combination of the substances or in the form of a fixed combination, for example in a tablet or capsule.

Pharmaceutical formulations of the combination partner needed for this may either be obtained commercially as pharmaceutical compositions or may be formulated by the skilled man using conventional methods. The active substances which may be obtained commercially as pharmaceutical compositions are described in numerous places in the prior art, for example in the list of drugs that appears annually, the “Rote Liste®” of the federal association of the pharmaceutical industry, or in the annually updated compilation of manufacturers' information on prescription drugs known as the “Physicians' Desk Reference”.

Examples of antidiabetic combination partners are metformin; sulphonylureas such as glibenclamide, tolbutamide, glimepiride, glipizide, gliquidon, glibornuride and gliclazide; nateglinide; repaglinide; thiazolidinediones such as rosiglitazone and pioglitazone; PPAR gamma modulators such as metaglidases; PPAR-gamma agonists such as GI 262570; PPAR-gamma antagonists; PPAR-gamma/alpha modulators such as tesaglitazar, muraglitazar, aleglitazar, indeglitazar and KRP297; PPAR-gamma/alpha/delta modulators; AMPK-activators such as AICAR; acetyl-CoA carboxylase (ACC1 and ACC2) inhibitors; diacylglycerol-acetyltransferase (DGAT) inhibitors; pancreatic beta cell GCRP agonists such as SMT3-receptor-agonists and GPR119; 11-HSD-inhibitors; FGF19 agonists or analogues; alpha-glucosidase blockers such as acarbose, voglibose and miglitol; alpha2-antagonists; insulin and insulin analogues such as human insulin, insulin lispro, insulin glusilin, r-DNA-insulinaspart, NPH insulin, insulin detemir, insulin zinc suspension and insulin glargin; Gastric inhibitory Peptide (GIP); amylin and amylin analogues (e.g. pramlintide or davalintide); GLP-1 and GLP-1 analogues such as Exendin-4, e.g. exenatide, exenatide LAR, liraglutide, taspoglutide, lixisenatide (AVE-0010), LY-2428757 (a PEGylated version of GLP-1), LY-2189265 (GLP-1 analogue linked to IgG4-Fc heavy chain), semaglutide or albiglutide; SGLT2-inhibitors such as e.g. dapagliflozin, sergliflozin (KGT-1251), atigliflozin, canagliflozin or (1S)-1,5-anhydro-1-[3-(1-benzothiophen-2-ylmethyl)-4-fluorophenyl]-D-glucitol; inhibitors of protein tyrosine-phosphatase (e.g. trodusquemine); inhibitors of glucose-6-phosphatase; fructose-1,6-bisphosphatase modulators; glycogen phosphorylase modulators; glucagon receptor antagonists; phosphoenolpyruvatecarboxykinase (PEPCK) inhibitors; pyruvate dehydrogenasekinase (PDK) inhibitors; inhibitors of tyrosine-kinases (50 mg to 600 mg) such as PDGF-receptor-kinase (cf. EP-A-564409, WO 98/35958, U.S. Pat. No. 5,093,330, WO 2004/005281, and WO 2006/041976); glucokinase/regulatory protein modulators incl. glucokinase activators; glycogen synthase kinase inhibitors; inhibitors of the SH2-domain-containing inositol 5-phosphatase type 2 (SHIP2); IKK inhibitors such as high-dose salicylate; JNK1 inhibitors; protein kinase C-theta inhibitors; beta 3 agonists such as ritobegron, YM 178, solabegron, talibegron, N-5984, GRC-1087, rafabegron, FMP825; aldosereductase inhibitors such as AS 3201, zenarestat, fidarestat, epalrestat, ranirestat, NZ-314, CP-744809, and CT-112; SGLT-1 or SGLT-2 inhibitors; KV 1.3 channel inhibitors; GPR40 modulators; SCD-1 inhibitors; CCR-2 antagonists; dopamine receptor agonists (bromocriptine mesylate [Cycloset]); sirtuin stimulants; and other DPP IV inhibitors.

Metformin is usually given in doses varying from about 500 mg to 2000 mg up to 2500 mg per day using various dosing regimens from about 100 mg to 500 mg or 200 mg to 850 mg (1-3 times a day), or about 300 mg to 1000 mg once or twice a day, or delayed-release metformin in doses of about 100 mg to 1000 mg or preferably 500 mg to 1000 mg once or twice a day or about 500 mg to 2000 mg once a day. Particular dosage strengths may be 250, 500, 625, 750, 850 and 1000 mg of metformin hydrochloride.

For children 10 to 16 years of age, the recommended starting dose of metformin is 500 mg given once daily. If this dose fails to produce adequate results, the dose may be increased to 500 mg twice daily. Further increases may be made in increments of 500 mg weekly to a maximum daily dose of 2000 mg, given in divided doses (e.g. 2 or 3 divided doses).

Metformin may be administered with food to decrease nausea.

A dosage of pioglitazone is usually of about 1-10 mg, 15 mg, 30 mg, or 45 mg once a day.

Rosiglitazone is usually given in doses from 4 to 8 mg once (or divided twice) a day (typical dosage strengths are 2, 4 and 8 mg).

Glibenclamide (glyburide) is usually given in doses from 2.5-5 to 20 mg once (or divided twice) a day (typical dosage strengths are 1.25, 2.5 and 5 mg), or micronized glibenclamide in doses from 0.75-3 to 12 mg once (or divided twice) a day (typical dosage strengths are 1.5, 3, 4.5 and 6 mg).

Glipizide is usually given in doses from 2.5 to 10-20 mg once (or up to 40 mg divided twice) a day (typical dosage strengths are 5 and 10 mg), or extended-release glibenclamide in doses from 5 to 10 mg (up to 20 mg) once a day (typical dosage strengths are 2.5, 5 and 10 mg).

Glimepiride is usually given in doses from 1-2 to 4 mg (up to 8 mg) once a day (typical dosage strengths are 1, 2 and 4 mg).

A dual combination of glibenclamide/metformin is usually given in doses from 1.25/250 once daily to 10/1000 mg twice daily. (typical dosage strengths are 1.25/250, 2.5/500 and 5/500 mg).

A dual combination of glipizide/metformin is usually given in doses from 2.5/250 to 10/1000 mg twice daily (typical dosage strengths are 2.5/250, 2.5/500 and 5/500 mg).

A dual combination of glimepiride/metformin is usually given in doses from 1/250 to 4/1000 mg twice daily.

A dual combination of rosiglitazone/glimepiride is usually given in doses from 4/1 once or twice daily to 4/2 mg twice daily (typical dosage strengths are 4/1, 4/2, 4/4, 8/2 and 8/4 mg).

A dual combination of pioglitazone/glimepiride is usually given in doses from 30/2 to 30/4 mg once daily (typical dosage strengths are 30/4 and 45/4 mg).

A dual combination of rosiglitazone/metformin is usually given in doses from 1/500 to 4/1000 mg twice daily (typical dosage strengths are 1/500, 2/500, 4/500, 2/1000 and 4/1000 mg).

A dual combination of pioglitazone/metformin is usually given in doses from 15/500 once or twice daily to 15/850 mg thrice daily (typical dosage strengths are 15/500 and 15/850 mg).

The non-sulphonylurea insulin secretagogue nateglinide is usually given in doses from 60 to 120 mg with meals (up to 360 mg/day, typical dosage strengths are 60 and 120 mg); repaglinide is usually given in doses from 0.5 to 4 mg with meals (up to 16 mg/day, typical dosage strengths are 0.5, 1 and 2 mg). A dual combination of repaglinide/metformin is available in dosage strengths of 1/500 and 2/850 mg.

Acarbose is usually given in doses from 25 to 100 mg with meals. Miglitol is usually given in doses from 25 to 100 mg with meals.

Examples of combination partners that lower the lipid level in the blood are HMG-CoA-reductase inhibitors such as simvastatin, atorvastatin, lovastatin, fluvastatin, pravastatin, pitavastatin and rosuvastatin; fibrates such as bezafibrate, fenofibrate, clofibrate, gemfibrozil, etofibrate and etofyllinclofibrate; nicotinic acid and the derivatives thereof such as acipimox; PPAR-alpha agonists; PPAR-delta agonists; inhibitors of acyl-coenzyme A:cholesterolacyltransferase (ACAT; EC 2.3.1.26) such as avasimibe; cholesterol resorption inhibitors such as ezetimib; substances that bind to bile acid, such as cholestyramine, colestipol and colesevelam; inhibitors of bile acid transport; HDL modulating active substances such as D4F, reverse D4F, LXR modulating active substances and FXR modulating active substances; CETP inhibitors such as torcetrapib, JTT-705 (dalcetrapib) or compound 12 from WO 2007/005572 (anacetrapib); LDL receptor modulators; MTP inhibitors (e.g. lomitapide); and ApoB100 antisense RNA.

A dosage of atorvastatin is usually from 1 mg to 40 mg or 10 mg to 80 mg once a day

Examples of combination partners that lower blood pressure are beta-blockers such as atenolol, bisoprolol, celiprolol, metoprolol and carvedilol; diuretics such as hydrochlorothiazide, chlortalidon, xipamide, furosemide, piretanide, torasemide, spironolactone, eplerenone, amiloride and triamterene; calcium channel blockers such as amlodipine, nifedipine, nitrendipine, nisoldipine, nicardipine, felodipine, lacidipine, lercanipidine, manidipine, isradipine, nilvadipine, verapamil, gallopamil and diltiazem; ACE inhibitors such as ramipril, lisinopril, cilazapril, quinapril, captopril, enalapril, benazepril, perindopril, fosinopril and trandolapril; as well as angiotensin II receptor blockers (ARBs) such as telmisartan, candesartan, valsartan, losartan, irbesartan, olmesartan and eprosartan.

A dosage of telmisartan is usually from 20 mg to 320 mg or 40 mg to 160 mg per day.

Examples of combination partners which increase the HDL level in the blood are Cholesteryl Ester Transfer Protein (CETP) inhibitors; inhibitors of endothelial lipase; regulators of ABC1; LXRalpha antagonists; LXRbeta agonists; PPAR-delta agonists; LXRalpha/beta regulators, and substances that increase the expression and/or plasma concentration of apolipoprotein A-I.

Examples of combination partners for the treatment of obesity are sibutramine; tetrahydrolipstatin (orlistat); alizyme (cetilistat); dexfenfluramine; axokine; cannabinoid receptor 1 antagonists such as the CB1 antagonist rimonobant; MCH-1 receptor antagonists; MC4 receptor agonists; NPY5 as well as NPY2 antagonists (e.g. velneperit); beta3-AR agonists such as SB-418790 and AD-9677; 5HT2c receptor agonists such as APD 356 (lorcaserin); myostatin inhibitors; Acrp30 and adiponectin; steroyl CoA desaturase (SCD1) inhibitors; fatty acid synthase (FAS) inhibitors; CCK receptor agonists; Ghrelin receptor modulators; Pyy 3-36; orexin receptor antagonists; and tesofensine; as well as the dual combinations bupropion/naltrexone, bupropion/zonisamide, topiramate/phentermine and pramlintide/metreleptin.

Examples of combination partners for the treatment of atherosclerosis are phospholipase A2 inhibitors; inhibitors of tyrosine-kinases (50 mg to 600 mg) such as PDGF-receptor-kinase (cf. EP-A-564409, WO 98/35958, U.S. Pat. No. 5,093,330, WO 2004/005281, and WO 2006/041976); oxLDL antibodies and oxLDL vaccines; apoA-1 Milano; ASA; and VCAM-1 inhibitors.

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

Further embodiments, features and advantages of the present invention may become apparent from the following examples. The following examples serve to illustrate, by way of example, the principles of the invention without restricting it.

### PHARMACOLOGICAL EXAMPLES

The following examples show the beneficial effect on glycemic control of the DPP-4 inhibitors or combinations according to the present invention.

### Example 1

According to a first example an oral glucose tolerance test is performed in overnight fasted male Zucker Diabetic Fatty (ZDF) rats (ZDF/Crl-Lep^{fa}). A pre-dose blood sample is obtained by tail bleed. Blood glucose is measured with a glucometer, and the animals are randomized for blood glucose (n=5/group). Subsequently, the groups receive a single oral administration of either vehicle alone (0.5% aqueous hydroxyethylcellulose containing 3 mM HCl and 0.015% Polysorbat 80) or vehicle containing either the DPP-4 inhibitor or the second or third antidiabetic agent or the combination of the DPP-4 inhibitor plus the second plus, optionally, the third antidiabetic agent. Alternatively, the test can also be performed after multiple administrations of the respective drugs to account for anti-diabetic effects that need longer to become evident like in the case of thiazolidindiones. The animals receive an oral glucose load (2 g/kg) 30 min after compound administration. Blood glucose is measured in tail blood 30 min, 60 min, 90 min, 120 min, and 180 min after the glucose challenge. Glucose excursion is quantified by calculating the reactive glucose AUC. The data are presented as mean±SEM. The two-sided unpaired Student t-test is used for statistical comparison of the control group and the active groups.

### Example 2

According to a second example an oral glucose tolerance test is performed in overnight fasted male Sprague Dawley rats (Crl:CD(SD)) with a body weight of about 200 g. A pre-dose blood sample is obtained by tail bleed. Blood glucose is measured with a glucometer, and the animals are randomized for blood glucose (n=5/group). Subsequently, the groups receive a single oral administration of either vehicle alone (0.5% aqueous hydroxyethylcellulose containing 0.015% Polysorbat 80) or vehicle containing either the DPP-4 inhibitor or the second or third antidiabetic agent or the combination of the DPP-4 inhibitor plus the second plus, optionally, the third antidiabetic agent. Alternatively the groups receive a single oral administration of either vehicle alone or vehicle containing either the DPP-4 inhibitor or the second antidiabetic agent plus the third antidiabetic agent or the combination of the DPP-4 inhibitor plus the second antidiabetic agent plus the third antidiabetic agent. Alternatively, the test can also be performed after multiple administrations of the respective drugs to account for anti-diabetic effects that need longer to become evident like in the case of thiazolidindiones. The animals receive an oral glucose load (2 g/kg) 30 min after compound administration. Blood glucose is measured in tail blood 30 min, 60 min, 90 min, and 120 min after the glucose challenge. Glucose excursion is quantified by calculating the reactive glucose AUC. The data are presented as mean±S.E.M. Statistical comparisons are conducted by Student's t test.

### Example 3: Treatment of Pre-Diabetes

The efficacy of a pharmaceutical composition or combination according to the invention in the treatment of pre-diabetes characterised by pathological fasting glucose and/or impaired glucose tolerance can be tested using clinical studies. In studies over a shorter period (e.g. 2-4 weeks) the success of the treatment is examined by determining the fasting glucose values and/or the glucose values after a meal or after a loading test (oral glucose tolerance test or food tolerance test after a defined meal) after the end of the period of therapy for the study and comparing them with the values before the start of the study and/or with those of a placebo group. In addition, the fructosamine value can be determined before and after therapy and compared with the initial value and/or the placebo value. A significant drop in the fasting or non-fasting glucose levels demonstrates the efficacy of the treatment. In studies over a longer period (12 weeks or more) the success of the treatment is tested by determining the HbA1c value, by comparison with the initial value and/or with the value of the placebo group. A significant change in the HbA1c value compared with the initial value and/or the placebo value demonstrates the efficacy of the DPP-4 inhibitors or combinations according to the present invention for treating pre-diabetes.

### Example 4: Preventing Manifest Type 2 Diabetes

Treating patients with pathological fasting glucose and/or impaired glucose tolerance (pre-diabetes) is also in pursuit of the goal of preventing the transition to manifest type 2 diabetes. The efficacy of a treatment can be investigated in a comparative clinical study in which pre-diabetes patients are treated over a lengthy period (e.g. 1-5 years) with either a pharmaceutical composition or combination according to this invention or with placebo or with a non-drug therapy or other medicaments. During and at the end of the therapy, by determining the fasting glucose and/or a loading test (e.g. oGTT), a check is made to determine how many patients exhibit manifest type 2 diabetes, i.e. a fasting glucose level of >125 mg/dl and/or a 2 h value according to oGTT of >199 mg/dl. A significant reduction in the number of patients who exhibit manifest type 2 diabetes when treated with a DPP-4 inhibitor or combination according to the present invention as compared to one of the other forms of treatment, demonstrates the efficacy in preventing a transition from pre-diabetes to manifest diabetes.

### Example 5: Treatment of Type 2 Diabetes

Treating patients with type 2 diabetes with the pharmaceutical composition or combination according to the invention, in addition to producing an acute improvement in the glucose metabolic situation, prevents a deterioration in the metabolic situation in the long term. This can be observed is patients are treated for a longer period, e.g. 3 months to 1 year or even 1 to 6 years, with the pharmaceutical composition or combination according to the invention and are compared with patients who have been treated with other antidiabetic medicaments. There is evidence of therapeutic success compared with patients treated with other antidiabetic medicaments if no or only a slight increase in the fasting glucose and/or HbA1c value is observed. Further evidence of therapeutic success is obtained if a significantly smaller percentage of the patients treated with a pharmaceutical composition or combination according to the invention, compared with patients who have been treated with other medicaments, undergo a deterioration in the glucose metabolic position (e.g. an increase in the HbA1c value to >6.5% or >7%) to the point where treatment with an additional oral antidiabetic medicament or with insulin or with an insulin analogue is indicated.

### Example 6: Treatment of Insulin Resistance

In clinical studies running for different lengths of time (e.g. 2 weeks to 12 months) the success of the treatment is checked using a hyperinsulinaemic euglycaemic glucose clamp study. A significant rise in the glucose infusion rate at the end of the study, compared with the initial value or compared with a placebo group, or a group given a different therapy, proves the efficacy of a DPP-4 inhibitor, pharmaceutical composition or combination according to the present invention according to the invention in the treatment of insulin resistance.

### Example 7: Treatment of Hyperglycaemia

In clinical studies running for different lengths of time (e.g. 1 day to 24 months) the success of the treatment in patients with hyperglycaemia is checked by determining the fasting glucose or non-fasting glucose (e.g. after a meal or a loading test with oGTT or a defined meal). A significant fall in these glucose values during or at the end of the study, compared with the initial value or compared with a placebo group, or a group given a different therapy, proves the efficacy of a DPP-4 inhibitor, pharmaceutical composition or combination according to the present invention according to the invention in the treatment of hyperglycaemia.

### Example 8: Prevention of Micro- or Macrovascular Complications

The treatment of type 2 diabetes or pre-diabetes patients with a DPP-4 inhibitor, pharmaceutical composition or combination according to the invention prevents or reduces or reduces the risk of developing microvascular complications (e.g. diabetic neuropathy, diabetic retinopathy, diabetic nephropathy, diabetic foot, diabetic ulcer) or macrovascular complications (e.g. myocardial infarct, acute coronary syndrome, unstable angina pectoris, stable angina pectoris, stroke, peripheral arterial occlusive disease, cardiomyopathy, heart failure, heart rhythm disorders, vascular restenosis). Type 2 diabetes or patients with pre-diabetes are treated long-term, e.g. for 1-6 years, with a pharmaceutical composition or combination according to the invention and compared with patients who have been treated with other antidiabetic medicaments or with placebo. Evidence of the therapeutic success compared with patients who have been treated with other antidiabetic medicaments or with placebo can be found in the smaller number of single or multiple complications. In the case of macrovascular events, diabetic foot and/or diabetic ulcer, the numbers are counted by anamnesis and various test methods. In the case of diabetic retinopathy the success of the treatment is determined by computer-controlled illumination and evaluation of the background to the eye or other ophthalmic methods. In the case of diabetic neuropathy, in addition to anamnesis and clinical examination, the nerve conduction rate can be measured using a calibrated tuning fork, for example. With regard to diabetic nephropathy the following parameters may be investigated before the start, during and at the end of the study: secretion of albumin, creatinine clearance, serum creatinin values, time taken for the serum creatinine values to double, time taken until dialysis becomes necessary.

### Example 9: Treatment of Metabolic Syndrome

The efficacy of a DPP-4 inhibitor, pharmaceutical composition or combination according to the present invention according to the invention can be tested in clinical studies with varying run times (e.g. 12 weeks to 6 years) by determining the fasting glucose or non-fasting glucose (e.g. after a meal or a loading test with oGTT or a defined meal) or the HbA1c value. A significant fall in these glucose values or HbA1c values during or at the end of the study, compared with the initial value or compared with a placebo group, or a group given a different therapy, proves the efficacy of an active substance or combination of active substances in the treatment of Metabolic Syndrome. Examples of this are a reduction in systolic and/or diastolic blood pressure, a lowering of the plasma triglycerides, a reduction in total or LDL cholesterol, an increase in HDL cholesterol or a reduction in weight, either compared with the starting value at the beginning of the study or in comparison with a group of patients treated with placebo or a different therapy.

### Example 10a: Prevention of NODAT and/or PTMS, and NODAT/PTMS Associated Complications

Treatment of patients after organ transplantation with the pharmaceutical composition according to the invention prevents the development of NODAT and/or PTMS, and associated complications. The efficacy of the treatment can be investigated in a comparative clinical study in which patients before or immediately after transplantation are treated over a lengthy period (e.g. 1-5 years) with either a pharmaceutical composition according to this intervention or with a placebo or with a non-drug therapy or other medicaments. During and at the end of the therapy, the incidence of NODAT, PTMS, micro- and macrovascular complications, graft rejection, infection and death will be assessed. A significant reduction in the number of patients experiencing these complications demonstrates the efficacy in preventing development of NODAT, PTMS, and associated complications.

### Example 10b: Treatment of NODAT and/or PTMS with Prevention, Delay or Reduction of Associated Complications

Treatment of patients with NODAT and/or PTMS with the pharmaceutical composition according to the invention prevents, delays or reduces the development of NODAT/PTMS associated complications. The efficacy of the treatment can be investigated in a comparative clinical study in which patients with NODAT and/or PTMS are treated over a lengthy period (e.g. 1-5 years) with either a pharmaceutical composition according to this intervention or with a placebo or with a non-drug therapy or other medicaments. During and at the end of the therapy, the incidence of micro- and macrovascular complications, graft rejection, infection and death will be assessed. A significant reduction in the number of patients experiencing these complications demonstrates the efficacy in preventing, delaying or reducing the development of NODAT and/or PTMS associated complications.

### Example 12: Treatment of Hyperuricemia

Patients with elevated levels of uric acid above the normal range (above 8.3 mg/dL or 494 μmol/L) or patients with a history of gout or gouty arthritis with a uric acid level greater than 6.0 mg/dL or 357 μmol/L have a significant risk of future episodes of gout or gouty arthritis as well as having an increased risk of cardiovascular disease. Therapy may be provided with the objective of lowering serum levels of uric acid as a means of preventing future episodes or flare-ups of gout or gouty arthritis. Additionally, lowering serum uric acid levels may reduce the risk of cardiovascular disease. For this purpose patients with an elevated uric acid level or a history of gout or gouty arthritis are treated either with a pharmaceutical composition according to the invention or with placebo or with a non-drug therapy or with other medicaments, over a lengthy period (e.g. 6 months to 4 years). During and at the end of the treatment a check is carried out by determining the serum uric acid level and the number of episodes of gout or gouty arthritis occurences. A reduction in uric acid below 6.0 mg/dL and/or fewer episodes of gout or gouty arthritis occurrence when treated with a pharmaceutical composition according to the invention compared with a different type of therapy, is proof of the efficacy of a pharmaceutical composition in preventing episodic gout or gouty arthritis or treating hyperuricemia.

### Example 13: Linagliptin Improves Hepatic Steatosis in Rodent Models

Hepatic steatosis is a hallmark of patients with type 2 diabetes and underlies the pathogenesis of non-alcoholic fatty liver disease (NAFLD). Linagliptin is a selective and non-renal excreted inhibitor of dipeptidyl peptidase-4 (DPP-4). In a model of diet-induced obesity (DIO, fed for 2 or 3 months), the effect of 4 weeks therapy with linagliptin (3 and 30 mg/kg/d, n=10) is investigated. Liver lipid content is detected by magnetic resonance spectroscopy (MRS) in vivo and ex vivo by analysis of liver triglycerides. DPP-4 activity is inhibited significantly (p<0.001) by 67%-80% and 79%-89% (3 and 30 mg/kg, resp.) compared to controls. Blood glucose levels following an OGTT (AUC) are significantly (p<0.01) suppressed ranging from 16%-20% (3 mg/kg/d) and 20%-26% (30 mg/kg/d). Liver fat content (MRS detection) is reduced significantly, except in the 3 mg/kg dose in the 2 month fed DIO mice. A significant reduction of liver fat content (MRS) is visible as early as 2 weeks on treatment. The correlation between liver lipid content as measured by MRS and hepatic triglyceride levels as measured ex vivo is r2=0.565 (p<0.0001).

In a 3^{rd} study ob/ob mice are analysed after 14 d of linagliptin treatment (3 mg/kg/d) and blinded histological scoring is performed (severity and grade of fat content, markers of inflammation). DPP-4 activity is inhibited by 80% and blood glucose AUC reduction is 25%. The histological score reveals less hepatic steatosis and inflammation in the linagliptin group (2.2±0.13, n=9, p<0.01) vs. control (3±0.18, n=10). In conclusion, linagliptin significantly reduces liver fat content and histological NAFLD in two different rodent models, likely due to a liver specific insulin sensitizing effect. The reversal of hepatic steatosis supports the use of linagliptin in patients with type 2 diabetes as well as NAFLD.

### Examples of Formulations

The following examples of formulations, which may be obtained analogously to methods known in the art, serve to illustrate the present invention more fully without restricting it to the contents of these examples. The term “active substance” denotes one or more compounds according to the invention, i.e. denotes a DPP-4 inhibitor or a second or third antidiabetic compound according to this invention or a combination of two or three of said active ingredients, for example selected from the combinations as listed in the Table 1 or 2. Additional suitable formulations for the DPP-4 inhibitor linagliptin may be those formulations disclosed in the application WO 2007/128724, the disclosure of which is incorporated herein in its entirety. Additional suitable formulations for the other DPP-4 inhibitors may be those formulations which are available on the market, or formulations described in the patent applications cited above in paragraph “background of the invention”, or those described in the literature, for example as disclosed in current issues of “Rote Liste®” (Germany) or of “Physician's Desk Reference”.

### Example 1

Dry ampoule containing 75 mg of active substance per 10 ml

Composition:

| | | |
|---|---|---|
| Active substance | 75.0 | mg |
| Mannitol | 50.0 | mg |
| water for injections | ad 10.0 | ml |

Preparation:

Active substance and mannitol are dissolved in water. After packaging the solution is freeze-dried. To produce the solution ready for use, the product is dissolved in water for injections.

### Example 2

Dry ampoule containing 35 mg of active substance per 2 ml

Composition:

| | | |
|---|---|---|
| Active substance | 35.0 | mg |
| Mannitol | 100.0 | mg |
| water for injections | ad 2.0 | ml |

Preparation:

Active substance and mannitol are dissolved in water. After packaging, the solution is freeze-dried.

To produce the solution ready for use, the product is dissolved in water for injections.

### Example 3

Tablet containing 50 mg of active substance

Composition:

| | | | |
|---|---|---|---|
| (1) | Active substance | 50.0 | mg |
| (2) | Mannitol | 98.0 | mg |
| (3) | Maize starch | 50.0 | mg |
| (4) | Polyvinylpyrrolidone | 15.0 | mg |
| (5) | Magnesium stearate | 2.0 | mg |
| | | 215.0 | mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 9 mm.

### Example 4

Tablet containing 350 mg of active substance

Preparation:

| | | | |
|---|---|---|---|
| (1) | Active substance | 350.0 | mg |
| (2) | Mannitol | 136.0 | mg |
| (3) | Maize starch | 80.0 | mg |
| (4) | Polyvinylpyrrolidone | 30.0 | mg |
| (5) | Magnesium stearate | 4.0 | mg |
| | | 600.0 | mg |

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 12 mm.

### Example 5

Capsules containing 50 mg of active substance

Composition:

| | | | |
|---|---|---|---|
| (1) | Active substance | 50.0 | mg |
| (2) | Dried maize starch | 58.0 | mg |
| (3) | Mannitol | 50.0 | mg |
| (4) | Magnesium stearate | 2.0 | mg |
| | | 160.0 | mg |

Preparation:

(1) is triturated with (3). This trituration is added to the mixture of (2) and (4) with vigorous mixing. This powder mixture is packed into size 3 hard gelatin capsules in a capsule filling machine.

### Example 6

Capsules containing 350 mg of active substance

Composition:

| | | | |
|---|---|---|---|
| (1) | Active substance | 350.0 | mg |
| (2) | Dried maize starch | 46.0 | mg |
| (3) | Mannitol | 30.0 | mg |
| (4) | Magnesium stearate | 4.0 | mg |
| | | 430.0 | mg |

Preparation:

(1) is triturated with (3). This trituration is added to the mixture of (2) and (4) with vigorous mixing. This powder mixture is packed into size 0 hard gelatin capsules in a capsule filling machine.

1. A method for treating a patient showing or having an increased risk for hepatic disease, the method comprising administering to the patient:
(a) linagliptin, and, optionally,
(b) a second antidiabetic agent selected from the group consisting of biguanides, thiazolidindiones, sulfonylureas, glinides, inhibitors of alpha-glucosidase and GLP-1 analogues, and, optionally,
(c) a third antidiabetic agent being different from (b) selected from the group consisting of biguanides, thiazolidindiones, sulfonylureas, glinides, inhibitors of alpha-glucosidase and GLP-1 analogues,
or a pharmaceutically acceptable salt thereof.

2. The method of claim 1, wherein linagliptin is administered in a daily oral amount of 5 mg.

3. The method of claim 1, wherein the patient has type 2 diabetes.

4. A method for preventing, slowing, delaying, or treating a disease or condition attributed to an abnormal accumulation of liver or ectopic fat in a patient in need thereof, the method comprising administering to the patient:
(a) linagliptin, and, optionally,
(b) a second antidiabetic agent selected from the group consisting of biguanides, thiazolidindiones, sulfonylureas, glinides, inhibitors of alpha-glucosidase and GLP-1 analogues, and, optionally,
(c) a third antidiabetic agent being different from (b) selected from the group consisting of biguanides, thiazolidindiones, sulfonylureas, glinides, inhibitors of alpha-glucosidase and GLP-1 analogues,
or a pharmaceutically acceptable salt thereof.

5. The method of claim 4, wherein the disease or condition attributed to an abnormal accumulation of liver or ectopic fat is selected from the group consisting of general fatty liver, non-alcoholic fatty liver (NAFL), non-alcoholic steatohepatitis (NASH), hyperalimentation-induced fatty liver, diabetic fatty liver, alcoholic-induced fatty liver or toxic fatty liver, particularly non-alcoholic fatty liver disease (NAFLD), hepatic steatosis, non-alcoholic steatohepatitis (NASH) and liver fibrosis.

6. A method for preventing, slowing the progression, delaying, attenuating, treating or reversing hepatic steatosis, (hepatic) inflammation and/or an abnormal accumulation of liver fat in a patient in need thereof, the method comprising administering to the patient:
(a) linagliptin, and, optionally,
(b) a second antidiabetic agent selected from the group consisting of biguanides, thiazolidindiones, sulfonylureas, glinides, inhibitors of alpha-glucosidase and GLP-1 analogues, and, optionally,
(c) a third antidiabetic agent being different from (b) selected from the group consisting of biguanides, thiazolidindiones, sulfonylureas, glinides, inhibitors of alpha-glucosidase and GLP-1 analogues,
or a pharmaceutically acceptable salt thereof.
